(19) United States
(12) Patent Application Publication
(10) Pub. No.: US 2026/0224699 A1
(43) Pub. Date: Aug. 6, 2026

(54) PSGL1 ANTAGONISTS AND USES THEREOF

(71) Applicant: Sanford Burnham Prebys Medical Discovery Institute, La Jolla, CA (US)

(72) Inventors: Linda M. BRADLEY, San Diego, CA (US); Jennifer Lynn GRACIAS, Wyomissing, PA (US)

(21) Appl. No.: 19/150,881

(22) PCT Filed: Jan. 24, 2024
(86) PCT No.: PCT/US2024/012819

§ 371 Date: Jul. 24, 2025

Related U.S. Application Data
(60) Provisional application No. 63/481,570, filed on Jan. 25, 2023.
(60) Provisional application No. 63/488,931, filed on Mar. 7, 2023.

(51) Int. Cl. A61K 40/15; A61K 40/33; A61K 40/42; C07K 16/28

(52) U.S. Cl. CPC A61K 40/15; A61K 40/33; A61K 40/4202; A61K 40/428; C07K 16/2896

(57) ABSTRACT

Described and provided herein are compositions and methods useful for modulating PSGL1 (e.g., inhibiting PSGL1 negative regulation of the immune response). In certain instances, the inhibition and/or modulation of PSGL1 is achieved by utilizing a partially glycosylated PSGL1 fusion protein. As described herein, modulating (e.g., inhibiting) PSGL1 is useful for promoting (e.g., increasing) immune cell (e.g., T cell) function (e.g., inhibiting T cell exhaustion and/or increasing the number of CD8+ or CD4+ T cells). Moreover modulating (e.g., inhibiting) PSGL1 is useful for treating a tumor.

### CROSS REFERENCE

This application claims the benefit of U.S. Provisional Application No. 63/481,570, filed on Jan. 25, 2023, and U.S. Provisional Application No. 63/488,931, filed on Mar. 7, 2023, each of which is incorporated herein by reference in its entirety.

### SEQUENCE LISTING

The instant application contains a Sequence Listing which has been submitted in XML file format and is hereby incorporated by reference in its entirety. Said XML copy, created on Jan. 24, 2024, is named 42256-607-601 SEQ.XML and is 5,710 bytes in size.

### BACKGROUND

Lymphocytes in the immune system are critical for protecting against and clearing virally infected cells and tumor cells. However, when under conditions such as chronic viral infection or cancer, the sustained antigen exposure blunts the capacity of lymphocytes to generate a robust pro-inflammatory response, which lends the cellular environment more susceptible to uncontrolled division of foreign or harmful cells.

Immune checkpoint inhibitors are a type of cancer treatment that works by releasing the brakes on the immune system, allowing it to attack cancer cells more effectively. However, existing immune checkpoint inhibitors and the development of new immune checkpoint inhibitors have faced multiple challenges leading to treatment efficacy. Such challenges include, for example, target identification, immune-related adverse events, resistance, non-responsiveness, and the identification of responsive patients.

### SUMMARY

Described and provided herein are compositions and methods useful for treating a tumor via the modulation (e.g., inhibition) of PSGL1. PSGL1 is a negative regulator of immune function. Effective T cell function leads to the generation of effector T cells that mediate viral clearance, as well as memory T cells that confer protection against subsequent reinfection. As described herein, PSGL1 is a fundamental regulator of T cell down-regulation (e.g., exhaustion) that sustains expression of several inhibitory receptors, including PD1. Successfully targeting and modulating (e.g., inhibiting) PSGL1 utilizing the methods and compositions described herein allows for the activation of an effective immune response to a tumor and treatment of the tumor. Moreover, the successful targeting and modulating (e.g., inhibiting) of PSGL1 utilizing the methods and compositions described herein is useful for treating tumors that are generally resistant or non-responsive to other immune checkpoint therapies.

Provided herein are partially glycosylated PSGL1 polypeptides (e.g., recombinant partially glycosylated PSGL1 polypeptides) useful for modulating (e.g., inhibiting, preventing, and/or reducing, etc.) PSGL1 signaling and/or PSGL1 negative regulation (e.g., down regulation) of immune function. In some embodiments, a partially glycosylated PSGL1 polypeptide encompasses and refers to a PSGL1 polypeptide lacking one or more glycosylated amino acids. In certain embodiments, a reduction in glycosylation can be identified by a change (e.g., decrease) in average (mean) molecular weight or a decrease in the number (e.g., mean amount) of glycosylation. In some embodiments, the partially glycosylated PSGL1 polypeptide comprises a reduction in O-linked glycosylation (e.g., a reduced number of O-linked glycans). In some embodiments, the partially glycosylated PSGL1 polypeptide comprises a reduction in O-linked tetrasaccharide carbohydrates (e.g., a reduced number of tetrasaccharide carbohydrates). In certain embodiments, the tetrasaccharide carbohydrate is a Sailyl-Lewis-X tetra-saccharide.

Further provided herein are fusion polypeptides comprising: a partially glycosylated PSGL1 polypeptide; and an Fc polypeptide. In some embodiments, the Fc polypeptide is a variant Fc polypeptide (e.g., comprising one or more mutations relative to a wild-type Fc polypeptide). In some embodiments, the variant Fc polypeptide comprises one or more mutations that reduce ADCC and/or CDC (e.g., compared to a wild-type Fc polypeptide). Effector functions generally encompass and/or refer to those biological activities attributable to the Fc polypeptide region of an antibody, which vary with the antibody isotype.

Provided herein are modified anti-PSGL1 antibodies or antibody fragments, comprising a PSGL1-binding domain and a variant Fc polypeptide wherein: the PSGL1-binding domain binds PSGL1; and the variant Fc polypeptide comprises an Fc modification (e.g., one or more mutations compared to a wild-type Fc) that reduces binding of the variant Fc polypeptide to an effector cell (e.g., an APC). Also provided are modified anti-PSGL1 antibodies or antibody fragments, comprising a PSGL1-binding domain and a variant Fc polypeptide wherein: the PSGL1-binding domain binds PSGL1; and the variant Fc polypeptide comprises an Fc modification (e.g., one or more mutations compared to a wild-type Fc) that reduces ADCC and/or CDC (e.g., compared to a wild-type Fc polypeptide).

In some embodiments, the Fc modification is a deletion and/or removal of an Fc polypeptide. In such embodiments, the modified anti-PSGL1 antibody is a Fab′ antibody or a scFv antibody. In some embodiments, the variant Fc polypeptide exhibits lower binding to an Fc-receptor (e.g., Fc-gamma-receptor) when compared to a wild-type Fc polypeptide. In some embodiments, the variant Fc polypeptide comprises one or more mutations that reduce a ADCC and/or a CDC (e.g., compared to a wild-type Fc polypeptide).

Provided herein are methods of inhibiting PSGL1-mediated negative regulation of immune cells (e.g., T cells) or an immune response that are advantageous for use in treating a tumor. In some embodiments, provided herein are methods of treating a tumor and/or cancer in an individual, the methods comprising: administering a partially glycosylated PSGL1 polypeptide (e.g., a partially glycosylated PSGL1 fusion polypeptide) or a modified anti-PSGL1 antibody to the individual. In some embodiments, the method comprises administering a partially glycosylated PSGL1 polypeptide to the individual. In certain embodiments, the method comprises administering a partially glycosylated PSGL1 fusion polypeptide to the individual. In some embodiments, the method comprises administering a modified anti-PSGL1 antibody to the individual.

In certain embodiments, the tumor is a solid tumor. In certain embodiments, the cancer comprises a solid tumor. In certain embodiments, the cancer is melanoma (e.g., comprising a melanoma tumor). In certain embodiments, the cancer is mesothelioma (e.g., comprising a mesothelioma tumor).

In certain embodiments, the tumor and/or cancer is resistant and/or non-responsive to an immune checkpoint blockade immunotherapy (ICB) or PD1 blockade immunotherapy. For example, the non-PSGL1 targeting immune checkpoint blockade immunotherapy (ICB) or PD1 blockade immunotherapy does not decrease or inhibit tumor growth in a model (e.g., mouse model) having the resistant and/or non-responsive tumor and/or cancer.

In some embodiments, further provided herein are methods of inhibiting PSGL1-mediated negative regulation of immune cells (e.g., T cells) or an immune response that are advantageous for use in treating an infectious disease (e.g., virus). In some embodiments, provided herein are methods of treating an infection (e.g., viral infection) in an individual, the methods comprising: administering a partially glycosylated PSGL1 polypeptide or a modified anti-PSGL1 antibody to the individual. In certain embodiments, the method comprises administering a partially glycosylated PSGL1 polypeptide to the individual. In certain embodiments, the method comprises administering a partially glycosylated PSGL1 fusion polypeptide to the individual. In certain embodiments, the method comprises administering a modified anti-PSGL1 antibody to the individual. In certain embodiments, the infection comprises a viral infection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

This patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows that reducing PSGL1 activity increases the number of CD8+ T-cells expressing biomarkers of TCR activation.

FIG. 2 depicts flow cytometry histograms of data presented in FIG. 1.

FIG. 3 shows that decreasing PSGL1 increases cytokine production in CD4+ and CD8+ T-cells in response to anti-CD3-epsilon antibody stimulation.

FIG. 4 depicts flow cytometry plots demonstrating of Akt phosphorylation kinetics in WT and PSGL1−/−OT-I CD8+ T-cells that were stimulated with the SIINFEKL OVA peptide.

FIG. 5 shows that reducing PSGL1 causes prolonged and higher levels of phosphorylated Zap70, Erk1/2, and Akt compared to WT OT-I CD8+ T-cells.

FIG. 6 depicts immunoblot and quantification demonstrating increased and prolonged expression of pZap70, pErk1/2, and pAkt in PSGL1−/−OT-I CD8+ T-cells compared to WT following activation with an anti-CD3-epsilon antibody.

FIG. 7 depicts flow cytometry histograms demonstrating that more PSGL1−/−OT-I CD8+ T-cells express TCR activation biomarker CD44 2 days after activation with variant OVA peptides having altered TCR affinity.

FIG. 8 depicts flow cytometry histograms demonstrating that more PSGL1−/−OT-I CD8+ T-cells express TCR activation biomarker CD44 5 days after activation with variant OVA peptides in iT_{EX} and rested iT_{EFF} conditions.

FIG. 9 depicts FACS plots demonstrating that reduced PSGL1 activation increases IFN-gamma and TNF-alpha production in OT-I CD8+ T-cells after OVA peptide stimulation.

FIG. 10 depicts flow cytometry histograms of IFN-gamma and TNF-alpha production by WT and PSGL1−/−OT-I CD8+ T-cells shown in FIG. 9.

FIG. 11 depicts the frequency of IFN-gamma and TNF-alpha double-producing CD44+OT-I CD8+ T-cells under iT_{EFF} or iT_{EX} conditions following treatment with indicated OVA peptide.

FIG. 12 depicts the ratio of IFN-gamma and TNF-alpha double-producing PSGL1−/− to WT (KO/WT) CD44+OT-I CD8+ T-cells in iT_{EFF} or iT_{EX} conditions after treatment with indicated OVA peptide.

FIG. 13A depicts the frequency of IFN-gamma-producing WT and PSGL1−/− iT_{EFF} CD44+OT-I CD8+ T-cells cultured with indicated OVA peptide.

FIG. 13B depicts the ratio of IFN-gamma-producing WT and PSGL1−/−iT_{EFF} CD44+OT-I CD8+ T-cells cultured with indicated OVA peptide.

FIG. 13C depicts the frequency of TNF-alpha-producing WT and PSGL1−/−iT_{EFF} CD44+OT-I CD8+ T-cells cultured with indicated OVA peptide.

FIG. 13D depicts the ratio of TNF-alpha-producing WT and PSGL1−/−iT_{EFF} CD44+OT-I CD8+ T-cells cultured with indicated OVA peptide.

FIG. 14A shows a heat map of expression levels of phosphorylated proteins obtained from data collected by multiplexed mass spectrometry and phospho-proteomics analyses of WT versus PSGL1−/−OT-I (KO) CD8+ T-cells.

FIG. 14B depicts a table listing of enriched pathways of activated PSGL1−/− T-cells identified in analysis of multiplexes mass spectrometry data.

FIG. 14C shows a heat map of phosphorylated proteins associated with regulation of T-cell signaling/activation, proliferation and survival, calcium signaling, and metabolism in PSGL1−/− T-cells. Upregulation is denoted with “+”, downregulation is denoted with “−”.

FIG. 14D shows a gene enrichment analysis demonstrating activation of the ERK1/2 pathway in PSGL1−/−OT-I cells.

FIG. 15 depicts immunofluorescent flow cytometry images demonstrating colocalization of PSGL1 with CD3/TCR signaling complex in naïve CD8+ T-cells.

FIG. 16 depicts histograms showing the % positive similarity score from flow cytometry images of 10,000 CD8+ T-cells.

FIG. 17 depicts immunofluorescent flow cytometry images and % positivity score demonstrating colocalization of CD3/TCR complex and PSGL1 (FIG. 17A, 17B).

FIG. 18 demonstrates reduced open chromatin in ATAC-seq tracings within the Ubash3b gene region in PSGL1−/−OT-I CD8+ T-cells compared to WT.

FIG. 19 depicts principal component analysis (PCA) plot of individual WT (light gray) and KO (dark grey) ATAC-seq libraries.

FIG. 20 depicts a volcano plot showing significantly upregulated (right), downregulated (left), and unchanged chromatic accessibility in PSGL1−/−OT-I CD8+ T-cells compared to WT based upon ATAC-seq experiments.

FIG. 21 depicts ATAC-seq profile of reproducible chromatin accessibility in WT and PSGL1−/−OT-I CD8+ T-cell libraries.

FIG. 22 depicts pie charts showing the breakdown by type in all, upregulated, and downregulated peaks determined by analysis of ATAC-seq data.

FIG. 23A depicts a immunoblot of Sts-1 and beta-Actin loading control protein expression in naïve (top) or two-day activated (bottom) WT and PSGL1−/−OT-I CD8+ T-cells

FIG. 23B depicts quantification of Sts-1 and beta-Actin loading control protein expression in naïve (top) or two-day activated (bottom) WT and PSGL1−/−OT-I CD8+ T-cells from the immunoblot shown in FIG. 23A.

FIG. 24A depicts flow cytometry images of Sts-1, PSGL1, CD3, and Zap70 colocalization in individual naïve or 20-minute-activated WT and PSGL1−/−OT-I CD8+ T-cells. Within each colocalization, the left bar represents WT and the right bar represents PSGL1−/−OT-I CD8+ T-cells.

FIG. 24B depicts the similarity scores of Sts-1, PSGL1, CD3, and Zap70 colocalization in individual naïve WT and PSGL1−/−OT-I CD8+ T-cells.

FIG. 24C depicts the similarity scores of Sts-1, PSGL1, CD3, and Zap70 colocalization in individual 20-minute-activated WT and PSGL1−/−OT-I CD8+ T-cells.

FIG. 25A depicts the quantification of the extracellular acidification rate (ECAR) of WT and PSGL1−/−OT-I CD8+ T-cells after 3-day (iT_{EFF}) activation.

FIG. 25B depicts the quantification of the oxygen consumption rate (OCR) of WT and PSGL1−/−OT-I CD8+ T-cells after 3-day (iT_{EFF}) activation.

FIG. 25C shows the proton efflux rates (PER) calculated from the ECAR of WT and PSGL1−/−OT-I CD8+ T-cells in FIG. 25A.

FIG. 25D shows the glycolytic rates (glycoPER) calculated from the OCR of WT and PSGL1−/−OT-I CD8+ T-cells in FIG. 25B.

FIG. 26A depicts the quantification of the extracellular acidification rate (ECAR) of WT and PSGL1−/−OT-I CD8+ T-cells after 5-day (iT_{EX}) activation.

FIG. 26B depicts the quantification of the oxygen consumption rate (OCR) of WT and PSGL1−/−OT-I CD8+ T-cells after chronic 5-day (iT_{EX}) activation.

FIG. 26C shows the proton efflux rates (PER) calculated from the ECAR of WT and PSGL1−/−OT-I CD8+ T-cells after 5-day (iT_{EX}) activation in FIG. 26A.

FIG. 26D shows the glycolytic rates (glycoPER) calculated from the OCR of WT and PSGL1−/−OT-I CD8+ T-cells after 5-day (iT_{EX}) activation in FIG. 26B.

FIG. 27A depicts a histogram of 2-NBDG uptake in WT OT-I or PSGL1−/−OT-I CD8+ T-cells after 2-hour stimulation with the SIINFEKL peptide.

FIG. 27B shows a bar graph of quantified population averages of 2-NBDG uptake in OT-I or PSGL1−/−OT-I CD8+ T-cells after 2-hour stimulation with the SIINFEKL peptide.

FIG. 28 depicts increased average MFI of 2-NBDG uptake in YUMM1.5 melanoma tumor-infiltrating PSGL1−/−CD44+CD8+ T-cells compared to WT.

FIG. 29 illustrates the experimental design for generating single-cell RNA-sequencing (scRNA-seq) libraries.

FIG. 30 depicts CellLoupe analysis of single-cell RNA-sequencing (scRNA-seq) libraries of donor WT or donor PSGL1−/−OT-I CD8+ T-cells harvested from B16-OVA melanoma tumor-bearing mice.

FIG. 31 depicts Seurat analysis of single-cell RNA-sequencing (scRNA-seq) libraries of donor WT or donor PSGL1−/−OT-I CD8+ T-cells from B16-OVA melanoma tumor-bearing mice.

FIG. 32 shows SeqGeq analysis of cluster-specific gene expression of Gzmb and Ifng in WT and PSGL1−/−CD8+ T-cells.

FIG. 33 shows SeqGeqtSNE clustering of expression for indicated genes in WT and PSGL1−/−OT-I CD8+ T-cells (top) with library source color overall (bottom).

FIG. 34 shows flow cytometry plots and frequency quantification of TCF-1 and TOX expression versus CD44 expression in virus-specific WT (circles) or PSGL1−/−(squares) CD8+ T-cells after chronic LCMV CI13 infection.

FIG. 35 shows frequencies of TCF-1+ or TOX+, virus-specific GP (33-41)+ or NP (396-404)+, CD8+ T-cells in blood of WT (circles) or PSGL1−/−(squares) mice at 9 and 15 days post-infection.

FIG. 36 shows the relative per-cell expression (MFI) of TCF-1+ or TOX+ in virus-specific GP (33-41)+ or NP (396-404)+CD8+ T-cells in the blood of WT (circles) or PSGL1−/−(squares) mice on days 9 and 15 post-infection.

FIG. 37A depicts frequency quantification of Slamf6+ T-cells from donor WT P14 mice (white bars) and donor PSGL1−/−P14 mice (gray bars) in LCMV CI13-infected WT mice on day 60 post-infection, which demonstrates that decreasing PSGL1 signaling increases stem-cell-like T-cells (TSC).

FIG. 37B depicts frequency quantification of IL-7R-alpha+ T-cells from donor WT P14 mice (white bars) and donor PSGL1−/−P14 mice (gray bars) in LCMV CI13-infected WT mice on day 60 post-infection, which demonstrates that decreasing PSGL1 signaling increases stem-cell-like T-cells (TSC).

FIG. 38A depicts FACS plots and quantification of the frequency of TCF-1, TOX, IL-7R-alpha and KLRG1 expression in GP (33-41)+CD8+ T-cells in the blood of WT (circles) or PSGL1−/−(squares) mice at 9 days post infection (dpi).

FIG. 38B depicts the quantification of the frequency of TCF-1, TOX, IL-7R-alpha and KLRG1 expression in GP (33-41)+CD8+ T-cells in the blood of WT or PSGL1−/− mice at 9 dpi, based on the FACS data shown in FIG. 38A.

FIG. 39A shows the average per-cell MFI of TCF-1+ virus-specific GP (33-41)+ or NP (396-404)+CD8+ T-cells of LCMV C113-infected WT mice treated with control antibody or agonist PSGL1 antibody (4RA10).

FIG. 39B shows the average per-cell MFI of TOX+ virus-specific GP (33-41)+ or NP (396-404)+CD8+ T-cells of LCMV C113-infected WT mice treated with control antibody or agonist antibody (4RA10).

FIG. 40A depicts FACS plots of TCF-1 and TOX expression in CD44 hi donor WT and PSGL1−/−OT-I CD8+ T-cells in B16-OVA melanoma mice.

FIG. 40B depicts the population mean frequency quantification of TCF-1 and TOX expression in CD44 hi donor WT and PSGL1−/−OT-I CD8+ T-cells in B16-OVA melanoma mice, based on data shown in FIG. 40A.

FIG. 41 depicts flow cytometry plots and frequency quantification of IFN-gamma production in CD8+ T-cells of melanoma patients or healthy donors.

FIG. 42 depicts a histogram of CD 162/PSGL1 expression on CD8+ T-cells in 3 representative healthy donors and 3 representative melanoma patients.

FIG. 43 depicts flow cytometry plots of IFN-gamma and TNF-alpha production by CD8+ T-cells of healthy human donors under iT_{EFF}, iT_{EFF}+PSGL1 agonist, and iT_{EX} conditions.

FIG. 44A depicts dot plot of the frequencies of IFN-gamma and TNF-alpha double-producing CD8+ human memory T cells cultured under iT_{EFF}, iT_{EFF}+PSGL1 agonist, or iT_{EX} conditions.

FIG. 44B depicts dot plot of the frequencies of IFN-gamma and TNF-alpha double-producing CD8+ human memory T cells from donors with reduced cytokine production upon PSGL1 ligation.

FIG. 45 depicts increased EOMES/T-bet expression in CD8+ human memory T cells from donors with reduced cytokine production upon PSGL1 ligation.

FIG. 46 demonstrates that reducing PSGL1 activity slows cancerous mesothelioma tumor growth.

FIG. 47 demonstrates that Fab′ antibody-mediated PSGL1 blockade increases cytokine production during viral infection.

FIG. 48 demonstrates that Fab′ antibody-mediated PSGL1 blockade does not affect the frequency of NP (396-404)-specific CD8+ T-cells after 30 days post-infection with LCMV CI13.

FIG. 49 demonstrates that Fab′ antibody-mediated PSGL1 blockade in LCMV C113-infected mice increases the frequency of cytokine-producing CD8+ T-cells after restimulation with GP (33-41) peptide. Within each category, the left bar represents mice treated with IgG Fab′ and the right bar represents mice treated with 4RA10 Fab′.

FIG. 50 depicts FACS plots which demonstrate that Fab′ antibody-mediated PSGL1 blockade decreases PD1 and TIM-3 expression in virus-specific CD8+ T-cells.

FIG. 51 illustrates pie charts which demonstrate that Fab′ antibody-mediated PSGL1 blockade decreases expression of PD1, LAG3, and TIM-3 on NP (396-404)-specific CD8+ T-cells.

FIG. 52 demonstrates that rPSGL1-Fc-mediated PSGL1 blockade promotes T_{EFF} and decreases frequencies of PD1+ and TIM-3+ double-producing cells.

FIG. 53 depicts pie charts which demonstrate that rPSGL1-Fc-mediated PSGL1 blockade decreases T_{EX}-associated co-expression of inhibitory receptors (PD1, LAG3, TIM-3).

FIG. 54A demonstrates that rPSGL1-Fc-mediated PSGL1 blockade decreases TOX expression in adoptively transferred WT P14 CD8+ T-cells in LCMV C113-infected mice.

FIG. 54B demonstrates that rPSGL1-Fc-mediated PSGL1 blockade increases cytokine production in adoptively transferred WTP14 CD8+ T-cells in LCMV C113-infected mice.

FIG. 55 demonstrates that rPSGL1-Fc-mediated PSGL1 blockade reduces cancerous melanoma tumor growth rate.

FIG. 56 depicts histological images with anti-CD3 staining of YUMM1.5 tumor sections, demonstrating that rPSGL1-Fc-mediated PSGL1 blockade increases TCR activation in malignant melanoma tumors.

FIG. 57 demonstrates that rPSGL1-Fc-mediated PSGL1 blockade reduces growth rate of established melanoma tumors.

### DETAILED DESCRIPTION

Described and provided herein are compositions and methods useful for modulating PSGL1 (e.g., inhibiting PSGL1 negative regulation of the immune response). In certain instances, the inhibition and/or modulation of PSGL1 is achieved by utilizing a partially glycosylated PSGL1 fusion protein. In certain instances, the inhibition and/or modulation of PSGL1 is achieved by utilizing a modified anti-PSGL1 antibody comprising a PSGL1-binding domain and a variant Fc polypeptide (e.g., a dead Fc polypeptide having reduced binding to an effector cell). As described herein, modulating (e.g., inhibiting) PSGL1 is useful for promoting (e.g., increasing) immune cell (e.g., T cell) function (e.g., inhibiting T cell exhaustion and/or increasing the number of CD8+ or CD4+ T cells). Moreover modulating (e.g., inhibiting) PSGL1 is useful for treating a tumor.

### Partially Glycosylated PSGL1

Provided herein are partially glycosylated PSGL1 polypeptides (e.g., recombinant partially glycosylated PSGL1 polypeptides) useful for modulating (e.g., inhibiting, preventing, and/or reducing, etc.) PSGL1 signaling and/or PSGL1 negative regulation (e.g., down regulation) of immune function. A PSGL1 polypeptide (also known as: selectin P ligand, SELPLG, CD 162, and/or CLA) generally encompasses and refers to the polypeptide encoded by the PSGL1 gene (NCBI Gene ID 6404, Location: NC_000012.12 (108621895 . . . 108633894, complement), Ensembl: ENSG00000110876, and/or UniProtKB ID Q14242). In some embodiments, the PSGL1 is human PSGL1. A partially glycosylated PSGL1 polypeptide generally encompasses and refers to a PSGL1 polypeptide lacking one or more glycosylated amino acids. In certain embodiments, a reduction in glycosylation can be identified by a change (e.g., decrease) in average (mean) molecular weight or a decrease in the number (e.g., mean amount) of glycosylation. In some embodiments, the partially glycosylated PSGL1 polypeptide comprises a reduction in O-linked glycosylation (e.g., a reduced number of O-linked glycans). In some embodiments, the partially glycosylated PSGL1 polypeptide comprises a reduction in O-linked tetrasaccharide carbohydrates (e.g., a reduced number of tetrasaccharide carbohydrate). In certain embodiments, the tetrasaccharide carbohydrate is a Sailyl-Lewis-X tetrasaccharide. Sailyl-Lewis-X tetrasaccharide carbohydrates generally encompass and refers to a branched amino tetrasaccharide consisting of a sialyl residue, linked (2->3) to a galactosyl residue that in turn is linked (1->3) to a glucosaminyl residue, which is also carrying a fucosyl residue at the 4-position, having the formula/structure alpha-Neu5 Ac-(2->3)-beta-D-Gal-(1->3)-[alpha-L-Fuc-(1->4)]-D-GlcNAc (see, e.g., PubChem CID 10985677). In some embodiments, the glycosylation is at T57 (55-PETEPP-59) of SEQ ID NO: 1. In some embodiments, partially glycosylated PSGL1 polypeptide comprises a threonine mutation. In some embodiments, partially glycosylated PSGL1 polypeptide comprises a serine mutation. In some embodiments, the glycosylation is mediated by the fucosyl transferase 4, fucosyl transferase 7, or both fucosyl transferase 4 and 7.

In some embodiments, the partially glycosylated PSGL1 polypeptide comprises an ectodomain PSGL1 polypeptide having an average molecular weight less than an average molecular weight of an ectodomain PSGL1 polypeptide isolated from or expressed by a T-cell (e.g., a T-cell culture). In some embodiments, the partially glycosylated PSGL1 polypeptide comprises SEQ ID NO: 2 having an average molecular weight less than a control PSGL1 polypeptide comprising SEQ ID NO: 2 isolated from or expressed by a T-cell (e.g., a T-cell culture).

In some embodiments, the partially glycosylated PSGL1 polypeptide comprises an ectodomain PSGL1 polypeptide having a decreased amount (e.g., mean amount) of glycosylation compared to a control ectodomain PSGL1 polypeptide isolated from or expressed by a T-cell (e.g., a T-cell culture). In some embodiments, the partially glycosylated PSGL1 polypeptide comprises SEQ ID NO: 2 having a decreased amount (e.g., mean amount) of glycosylation compared to a control PSGL1 polypeptide comprising SEQ ID NO: 2 isolated from or expressed by a T-cell (e.g., a T-cell culture). In certain embodiments, the glycosylation is measured by mass spectrometry. In certain embodiments, the glycosylation is measured by molecular weight. In certain embodiments, the glycosylation is O-linked glycosylation. In certain embodiments, the glycosylation comprises a tetrasaccharide carbohydrate. In certain embodiments, the glycosylation comprises a Sailyl-Lewis-X tetrasaccharide. In certain embodiments, the glycosylation is mediated by the fucosyl transferase 4, fucosyl transferase 7, or both fucosyl transferase 4 and 7.

In some embodiments, the partially glycosylated PSGL1 is generated by cell lacking fucosyl transferase 4, fucosyl transferase 7, or both fucosyl transferase 4 and 7.

In some embodiments, the PSGL1 polypeptide (e.g., of the partially glycosylated PSGL1) comprises an amino acid sequence having at least 85% sequence identity to the ectodomain sequence of SEQ ID NO: 1. In some embodiments, the PSGL1 polypeptide (e.g., of the partially glycosylated PSGL1) comprises an amino acid sequence having at least 95% sequence identity to the ectodomain sequence of SEQ ID NO: 1. In some embodiments, the PSGL1 polypeptide (e.g., of the partially glycosylated PSGL1) comprises an amino acid sequence having at least 97% sequence identity to the ectodomain sequence of SEQ ID NO: 1. In some embodiments, the PSGL1 polypeptide (e.g., of the partially glycosylated PSGL1) comprises an amino acid sequence having at least 98% sequence identity to the ectodomain sequence of SEQ ID NO: 1. In some embodiments, the PSGL1 polypeptide (e.g., of the partially glycosylated PSGL1) comprises an amino acid sequence having at least 99% sequence identity to the ectodomain sequence of SEQ ID NO: 1. In some embodiments, the PSGL1 polypeptide (e.g., of the partially glycosylated PSGL1) comprises an amino acid sequence of the ectodomain sequence of SEQ ID NO: 1.

In some embodiments, the PSGL1 polypeptide (e.g., of the partially glycosylated PSGL1) comprises an amino acid sequence having at least 85% sequence identity to SEQ ID NO: 2. In some embodiments, the PSGL1 polypeptide (e.g., of the partially glycosylated PSGL1) comprises an amino acid sequence having at least 90% sequence identity to SEQ ID NO: 2. In some embodiments, the PSGL1 polypeptide (e.g., of the partially glycosylated PSGL1) comprises an amino acid sequence having at least 95% sequence identity to SEQ ID NO: 2. In some embodiments, the PSGL1 polypeptide (e.g., of the partially glycosylated PSGL1) comprises an amino acid sequence having at least 97% sequence identity to SEQ ID NO: 2. In some embodiments, the PSGL1 polypeptide (e.g., of the partially glycosylated PSGL1) comprises an amino acid sequence having at least 98% sequence identity to SEQ ID NO: 2. In some embodiments, the PSGL1 polypeptide (e.g., of the partially glycosylated PSGL1) comprises an amino acid sequence having at least 99% sequence identity to SEQ ID NO: 2. In some embodiments, the PSGL1 polypeptide (e.g., of the partially glycosylated PSGL1) comprises an amino acid sequence as set forth in SEQ ID NO: 2.

In some embodiments, the partially glycosylated PSGL1 polypeptide binds to a VISTA polypeptide (e.g., as measured by ELISA, surface plasmon resonance, isothermal titration calorimetry, or bio-layer interferometry). A VISTA polypeptide (also known as: V-set immunoregulatory receptor, VSIR, B7H5, GI24, B7-H5, Dies1, PD1H, SISP1, VISTA, pp 2135, C10orf54, DD1-alpha) generally encompasses and refers to the polypeptide encoded by the VISTA gene (NCBI Gene ID 64115, Location: NC_000010.11 (71747556 . . . 71773520, complement), Ensembl: ENSG00000107738, and/or UniProtKB: Q9H7M9). In some embodiments, the partially glycosylated PSGL1 polypeptide exhibits reduced (e.g., compared to a wild-type fully glycosylated PSGL1) or no binding to E-selectin and/or L-selectin. In some embodiments, reduces (e.g., competes with) binding of VISTA to a T-cell (e.g., as measured by flow cytometry, ELISA, or changes in gene expression such has reducing T cell expression of PD1).

### Partially Glycosylated PSGL1 Fusion Polypeptides

Further described and provided herein are fusion polypeptides comprising: a partially glycosylated PSGL1 polypeptide; and an Fc polypeptide. An Fc polypeptide generally encompasses and/or refers to a C-terminal region of an immunoglobulin heavy chain that contains at least a portion of the constant region. The term includes native Fc polypeptide sequences and variant Fc polypeptide sequences. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. The class of an Fc polypeptide refers to the type of constant domain or constant region possessed by its heavy chain. There are five major classes of antibodies: IgA, IgD, IgE, IgG, and IgM, and several of these can be further divided into subclasses (isotypes), e.g., IgG1, IgG2, IgG3, IgG4, IgA1, and IgA2.

In some embodiments, the Fc polypeptide comprises an IgG polypeptide. In some embodiments, the IgG polypeptide comprises an IgG1, IgG2, IgG3, or IgG4 polypeptide. In some embodiments, the Fc polypeptide comprises an amino acid sequence having at least 85% sequence identity to SEQ ID NO: 4. In some embodiments, the Fc polypeptide comprises an amino acid sequence having at least 90% sequence identity to SEQ ID NO: 4. In some embodiments, the Fc polypeptide comprises an amino acid sequence having at least 95% sequence identity to SEQ ID NO: 4. In some embodiments, the Fc polypeptide comprises an amino acid sequence having at least 97% sequence identity to SEQ ID NO: 4. In some embodiments, the Fc polypeptide comprises an amino acid sequence having at least 98% sequence identity to SEQ ID NO: 4. In some embodiments, the Fc polypeptide comprises an amino acid sequence having at least 99% sequence identity to SEQ ID NO: 4. In some embodiments, the Fc polypeptide comprises an amino acid as set forth in SEQ ID NO: 4.

In some embodiments, the Fc polypeptide is a variant Fc polypeptide (e.g., comprising one or more mutations relative to a wild-type Fc polypeptide). In some embodiments, the variant Fc polypeptide comprises one or more mutations that reduce ADCC and/or CDC (e.g., compared to a wild-type Fc polypeptide). Effector functions generally encompass and/or refer to those biological activities attributable to the Fc polypeptide region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation. In some embodiments, the antibodies described herein comprise mutations that reduce effector function.

### Modified Anti-PSGL1 Antibodies

Provided herein are modified anti-PSGL1 antibodies or antibody fragments, comprising a PSGL1-binding domain and a variant Fc polypeptide wherein: the PSGL1-binding domain binds PSGL1; and the variant Fc polypeptide comprises an Fc modification (e.g., one or more mutations compared to a wild-type Fc) that reduces binding of the variant Fc polypeptide to an effector cell (e.g., an APC). Also provided are modified anti-PSGL1 antibodies or antibody fragments, comprising a PSGL1-binding domain and a variant Fc polypeptide wherein: the PSGL1-binding domain binds PSGL1; and the variant Fc polypeptide comprises an Fc modification (e.g., one or more mutations compared to a wild-type Fc) that reduces ADCC and/or CDC (e.g., compared to a wild-type Fc polypeptide).

In some embodiments, the Fc modification is a deletion and/or removal of an Fc polypeptide. In such embodiments, the modified anti-PSGL1 antibody is a Fab′ antibody or a scFv antibody. In some embodiments, the variant Fc polypeptide exhibits lower binding to an Fc-receptor (e.g., Fc-gamma-receptor) when compared to a wild-type Fc polypeptide. In some embodiments, the variant Fc polypeptide comprises one or more mutations that reduce a ADCC and/or a CDC (e.g., compared to a wild-type Fc polypeptide). In some embodiments, the Fc modification comprises one or more sets of mutations (e.g., in IgG) selected from the group consisting of: aglycosylation (N297A/Q/G), L235A/G237A/E318A, L234A/L235A, S228P/L235E, G236R/L328R, S298G/T299A, L234F/L235E/P331S, H268Q/V309L/A330S/P331S, E233P/L234V/L235A/G236del/S267K, L234A/L235A/P329G, V234A/G237A/P238S/H268A/V309L/A330S/P331S, and L234F/L235E/D265A.

An antibody is used in the broadest sense, and generally encompasses and/or refer to various antibody structures, including but not limited to monoclonal antibodies, polyclonal antibodies, multi-specific antibodies (e.g., bispecific antibodies), and antibody fragments so long as they exhibit the desired antigen-binding activity. In some embodiments, an antibody or antibodies include intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. In some embodiments, an antibody or antibodies include genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In some embodiments, an antibody or antibodies encompass functional antibody fragments thereof. In some embodiments, an antibody or antibodies encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. In some embodiments, an antibody or antibodies include, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)2, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that can be present in minor amounts. A monoclonal antibody can comprise a human IgG1 constant region or a human IgG4 constant region.

A native antibody generally encompasses and/or refers to naturally occurring immunoglobulin molecules with varying structures. For example, native IgG antibodies are heterotetrameric glycoproteins of about 150,000 Daltons, composed of two identical light chains and two identical heavy chains that are disulfide bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody can be assigned to one of two types, called kappa (κ) and lambda (λ), based on the amino acid sequence of its constant domain.

A full-length antibody, intact antibody, and whole antibody are interchangeable, and generally include and/or refer to an antibody having a structure that is substantially similar to a native antibody structure or having heavy chains that contain an Fc region as defined herein.

A human consensus framework generally encompasses and/or refers to a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra. A humanized antibody encompasses and refers to a chimeric antibody comprising amino acid residues from non-human HVRs and amino acid residues from human framework regions (FRs). In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally encompasses at least a portion of an antibody constant region derived from a human antibody. A “humanized form” of an antibody, e.g., a non-human antibody, refers to an antibody that has undergone humanization.

A complementarity determining region or CDR, which are synonymous with hypervariable region or HVR, generally include and/or refer to regions of an antibody which are hypervariable in sequence and/or form structurally defined loops (hypervariable loops) and/or contain the antigen-contacting residues (antigen contacts). In some embodiments, complementarity determining regions or CDRs generally include and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

A framework region or FR generally includes and/or refers to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). In some embodiments, the framework regions are defined by the non-CDR sequences of a variable region sequence. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3): 657-70, (“Aho” numbering scheme); and Whitelegg N R and Rees A R, “WAM: an improved algorithm for modelling antibodies on the WEB,” Protein Eng. 2000 December; 13(12): 819-24 (“AbM” numbering scheme. In certain embodiments, the CDRs of the antibodies described herein can be defined by a method selected from Kabat, Chothia, IMGT, Aho, AbM, or combinations thereof. In some embodiments, the CDRs and FRs are defined by and/or according to a Kabat numbering scheme. In some embodiments, the CDRs and FRs are defined by and/or according to a Chothia numbering scheme. In some embodiments, the CDRs and FRs are defined by and/or according to a IMGT numbering scheme. In some embodiments, the CDRs and FRs are defined by and/or according to an EU numbering scheme.

The boundaries of a given CDR or FR, in certain instances, vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A human antibody generally encompasses and/or refers to an antibody which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

An acceptor human framework generally encompasses and/or refers to a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

A variable region or variable domain generally encompasses and/or refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively (See e.g., Portolano et al., J. Immunol. 150:880-887 (1993); Clarkson et al., Nature 352:624-628 (1991)).

Affinity generally encompasses and/or refers to the strength of the sum total of noncovalent interactions between a single binding site of a molecule (e.g., an antibody) and its binding partner (e.g., an antigen). Unless indicated otherwise, as used herein, binding affinity generally encompasses and refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described throughout.

An affinity matured antibody generally encompasses and/or refers to an antibody with one or more alterations in one or more hypervariable regions (HVRs), compared to a parent antibody which does not possess such alterations, such alterations resulting in an improvement in the affinity of the antibody for antigen.

An antibody that binds to the same epitope as a reference antibody encompasses and/or refers to an antibody that blocks binding of the reference antibody to its antigen in a competition assay by 50% or more, and conversely, the reference antibody blocks binding of the antibody to its antigen in a competition assay by 50% or more. An exemplary competition assay encompasses assays known in the art and the binding assays described herein.

A chimeric antibody generally encompasses and/or refers to an antibody in which a portion of the heavy and/or light chain is derived from a particular source or species, while the remainder of the heavy and/or light chain is derived from a different source or species. 1 The “class” of an antibody refers to the type of constant domain or constant region possessed by its heavy chain. There are five major classes of antibodies: IgA, IgD, IgE, IgG, and IgM, and several of these can be further divided into subclasses (isotypes), e.g., IgG1, IgG2, IgG3, IgG4, IgA1, and IgA2. The heavy chain constant domains that correspond to the different classes of immunoglobulins are called a, d, e, g, and m, respectively.

A monoclonal antibody generally encompasses and/or refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. In some embodiments, the modifier monoclonal indicates the character of the antibody as being obtained from a substantially homogeneous population of antibodies and is not to be construed as requiring production of the antibody by any particular method. For example, the monoclonal antibodies to be used in accordance with the present disclosure can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An isolated antibody generally encompasses and/or refers to an antibody that has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

### Methods

Provided herein are methods of inhibiting PSGL1-mediated negative regulation of immune cells (e.g., T cells) or an immune response that are advantageous for use in treating a tumor. Accordingly, in some embodiments, provided herein are methods of treating a tumor and/or cancer in an individual, the methods comprising: administering a partially glycosylated PSGL1 polypeptide (e.g., a partially glycosylated PSGL1 fusion polypeptide) or a modified anti-PSGL1 antibody to the individual. In some embodiments, the method comprises administering a partially glycosylated PSGL1 polypeptide to the individual. In certain embodiments, the method comprises administering a partially glycosylated PSGL1 fusion polypeptide to the individual. In some embodiments, the method comprises administering a modified anti-PSGL1 antibody to the individual.

In certain embodiments, the tumor is a solid tumor. In certain embodiments, the cancer comprises a solid tumor. In certain embodiments, the cancer is melanoma (e.g., comprising a melanoma tumor). In certain embodiments, the cancer is mesothelioma.

In certain embodiments, the tumor and/or cancer is resistant and/or non-responsive to an immune checkpoint blockade immunotherapy (ICB) or PD1 blockade immunotherapy. For example, the non-PSGL1 targeting immune checkpoint blockade immunotherapy (ICB) or PD1 blockade immunotherapy does not decrease or inhibit tumor growth in a model (e.g., mouse model) having the resistant and/or non-responsive tumor and/or cancer.

In some embodiments, further provided herein are methods of inhibiting PSGL1-mediated negative regulation of immune cells (e.g., T cells) or an immune response that are advantageous for use in treating an infectious disease (e.g., virus). Accordingly, in some embodiments, provided herein are methods of treating an infection (e.g., viral infection) in an individual, the methods comprising: administering a partially glycosylated PSGL1 polypeptide or a modified anti-PSGL1 antibody to the individual. In certain embodiments, the method comprises administering a partially glycosylated PSGL1 polypeptide to the individual. In certain embodiments, the method comprises administering a partially glycosylated PSGL1 fusion polypeptide to the individual. In certain embodiments, the method comprises administering a modified anti-PSGL1 antibody to the individual. In certain embodiments, the infection comprises a viral infection.

In some embodiments, provided herein are methods of inhibiting PSGL1-mediated negative regulation of immune cells (e.g., T cells) or an immune response. Accordingly, in some embodiments, provided herein are methods of reducing the negative regulation (e.g., exhaustion) of immune cells (e.g., T cells), the methods comprising: administering a partially glycosylated PSGL1 polypeptide or a modified anti-PSGL1 antibody to a plurality of cells, wherein the plurality of cells comprises the immune cells. In some embodiments, provided are methods of activating immune cells (e.g., T cells), the methods comprising: administering a partially glycosylated PSGL1 polypeptide or a modified anti-PSGL1 antibody to a plurality of cells, wherein the plurality of cells comprises the immune cells. In certain embodiments, the method comprises administering a partially glycosylated PSGL1 polypeptide. In certain embodiments, the method comprises administering a partially glycosylated PSGL1 fusion polypeptide. In certain embodiments, the method comprises administering a modified anti-PSGL1 antibody.

In certain embodiments, the immune cells comprise T-cells, NK cells, and or both T-cells and NK cells. In certain embodiments, the T cells comprise CD8+ T cells. In certain embodiments, the T cells comprise CD4+ T cells. In certain embodiments, the T cells comprise both CD8+ and CD4+ T cells.

In certain embodiments, administering the partially glycosylated PSGL1 polypeptide or the modified anti-PSGL1 antibody or antibody fragment increases the number of the immune cells relative to an equivalent plurality of cells and/or immune cells not treated with the partially glycosylated PSGL1 polypeptide or the modified anti-PSGL1 antibody or antibody fragment. In certain embodiments, administering the partially glycosylated PSGL1 polypeptide or the modified anti-PSGL1 antibody or antibody fragment decreases expression of PD1 of the immune cells relative to an equivalent plurality of cells or immune cells not treated with partially glycosylated PSGL1 polypeptide or the modified anti-PSGL1 antibody or antibody fragment. In certain embodiments, administering the partially glycosylated PSGL1 polypeptide or the modified anti-PSGL1 antibody or antibody fragment increases immune cell glycolysis (e.g., as measured by proton efflux rates) relative to an equivalent plurality of cells and/or immune cells not treated with the partially glycosylated PSGL1 polypeptide or the modified anti-PSGL1 antibody or antibody fragment.

In certain embodiments, administering the partially glycosylated PSGL1 polypeptide or the modified anti-PSGL1 antibody or antibody fragment: decreases binding of VISTA, P-selectin, E-selectin, L-selectin, CCL21, or CCL19 to PSGL1 on a T-cell; decreases at least one immunosuppressive biomarker within the plurality of cells (e.g., Sts-1 or TOX); increases glycolysis-mediated carbohydrate metabolism of the immune cells (e.g., extracellular acidification rate, oxygen consumption rate, proton efflux rate, or glycolytic rate); increases secretion of IL-2, IFNγ, or TNFα by the immune cells or within the plurality of cells; decreases expression of Sts-1 or TOX by the immune cells or within the plurality of cells; increases expression of at least one biomarker promoting T-cell receptor (TCR) signaling (e.g., CD25, CD44, CD44 hi, CD69, phosphorylated Akt, phosphorylated ERK1/2, phosphorylated Zap70, TCF-1, Lat, Stat5b, Myc, Nfatc2, Jun-b, Fos-1, mTor, Hif1a, Mki67, Pgam1, Aldoa, Eno1, or Ldha); or a combination thereof, relative to immune cells not treated with the partially glycosylated PSGL1 polypeptide or the modified anti-PSGL1 antibody or antibody fragment.

In certain embodiments, the immune cells and/or plurality of cells are in an individual. In certain embodiments, the individual has a tumor and/or cancer. In certain embodiments, the tumoris a solid tumor and/or the cancer comprises a solid tumor. In certain embodiments, the tumor and/or cancer is resistant and/or non-responsive to an immune checkpoint blockade immunotherapy (ICB) or PD1 blockade immunotherapy. In certain embodiments, the individual has a viral infection.

In some embodiments, the partially glycosylated PSGL1 polypeptide or the modified anti-PSGL1 antibody or antibody fragment is administer intravenously. IN certain embodiments, the partially glycosylated PSGL1 polypeptide or the modified anti-PSGL1 antibody or antibody fragment is administered as a pharmaceutical formulation. A pharmaceutical formulation generally encompasses and/or refers to a preparation which is in such form as to permit the biological activity of an active ingredient contained therein to be effective, and which contains no additional components which are unacceptably toxic to a subject to which the formulation would be administered. A pharmaceutically acceptable carrier generally encompasses and/or refers to an ingredient in a pharmaceutical formulation, other than an active ingredient, which is nontoxic to a subject. A pharmaceutically acceptable carrier encompasses, but is not limited to, a buffer, excipient, stabilizer, or preservative. In certain embodiments, the partially glycosylated PSGL1 polypeptide or the modified anti-PSGL1 antibody or antibody fragment of the current disclosure is administered suspended in a sterile and/or isotonic solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tw een 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

### Additional Definitions

As used herein, a peptide includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner that is similar to a naturally occurring amino acids. Amino acids are generally referred to herein by either their name, the commonly known three letter symbols, or by the one-letter symbols recommended by the IUPAC-IUB Biochemical Nomenclature Commission.

In some embodiments, the peptides (e.g., a PSGL1 polypeptide or a fusion polypeptide) comprise one or more naturally occurring amino acids. In some embodiments, the peptides (e.g., a PSGL1 polypeptide or a fusion polypeptide) consist of naturally occurring amino acids. As used herein, naturally occurring amino acids include and/or refer to amino acids which are generally found in nature and are not manipulated by man. In some embodiments, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, the peptides (e.g., a PSGL1 polypeptide or a fusion polypeptide) comprise a variant sequence. In some embodiments, amino acid substitutions can be made in the sequence of any of the polypeptides described herein, without necessarily decreasing or ablating its activity. Accordingly, in some embodiments, the variant sequence comprises one or more amino acid substitutions. In some embodiments, substitutions include conservative substitutions (e.g., substitutions with amino acids of comparable chemical characteristics). In some embodiments, a non-polar amino acid can be substituted and replaced with another non-polar amino acid, wherein non-polar amino acids include alanine, leucine, isoleucine, valine, glycine, proline, phenylalanine, tryptophan, and methionine. In some embodiments, a neutrally charged polar amino acid can be substituted and replaced with another neutrally charged polar amino acid, wherein neutrally charged polar amino acids include serine, threonine, cysteine, tyrosine, asparagine, and glutamine. In some embodiments, a positively charged amino acid can be substituted and replaced with another positively charged amino acid, wherein positively charged amino acids include arginine, lysine and histidine. In some embodiments, a negatively charged amino acid can be substituted and replaced with another negatively charged amino acid, wherein negatively charged amino acids include aspartic acid and glutamic acid. Examples of amino acid substitutions also include substituting an L-amino acid for its corresponding D-amino acid, substituting cysteine for homocysteine or other non-natural amino acids.

Percent (%) sequence identity with respect to a reference polypeptide sequence generally encompasses and refers to the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences can be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, individual is synonymous with patient and/or subject and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

As used herein, the term “inhibition” or “inhibiting” includes and/or refers to the reduction or suppression of a given condition, symptom, disorder, or disease, and/or a decrease in the baseline activity of a biological activity or process.

As used herein, the term “treating” or “treatment” of includes and/or refers to ameliorating the disease or disorder or symptoms thereof (e.g., slowing or arresting or reducing the development of the disease or at least one of the clinical symptoms thereof). In certain embodiments, “treating” or “treatment” also includes and/or refers to alleviating or ameliorating at least one physical and/or biological parameters including those which may not be discernible by the patient. In certain embodiments, “treating” or “treatment” includes and/or refers to modulating a disease, disorder, or biological process either physically, (e.g., stabilization of a discernible symptom), physiologically, (e.g., stabilization of a physical and/or biological parameter), or both. In certain embodiments, “treating” or “treatment” includes and/or refers to preventing or delaying the onset or development or progression of the disease or disorder. In certain embodiments, “treating” or “treatment” includes and/or refers to preventing or delaying or inhibiting the deterioration of (i) a healthy physiological state or (ii) a baseline physiological state (e.g., the progression of a disease or disorder).

As used herein, “a” or “an” when used in conjunction with the term “comprising” in the claims and/or the specification includes and/or refers to “one” and is also consistent with the meaning of “one or more”, “at least one”, and “one or more than one”. Similarly, the word “another” may mean at least a second or more.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used herein, the term “about” in the context of a given value or range includes and/or refers to a value or range that is within 20%, within 10%, and/or within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

As used herein, a “sample” includes and/or refers to any fluid or liquid sample which is being analyzed to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

As used herein, in any instance or embodiment described herein, “comprising” may be replaced with “consisting essentially of” and/or “consisting of”, used herein, in any instance or embodiment described herein, “comprises” may be replaced with “consists essentially of” and/or “consists of”.

### EXAMPLES

### Example 1: Reducing PSGL1 Increases the Magnitude and Duration of T-Cell Receptor Signaling

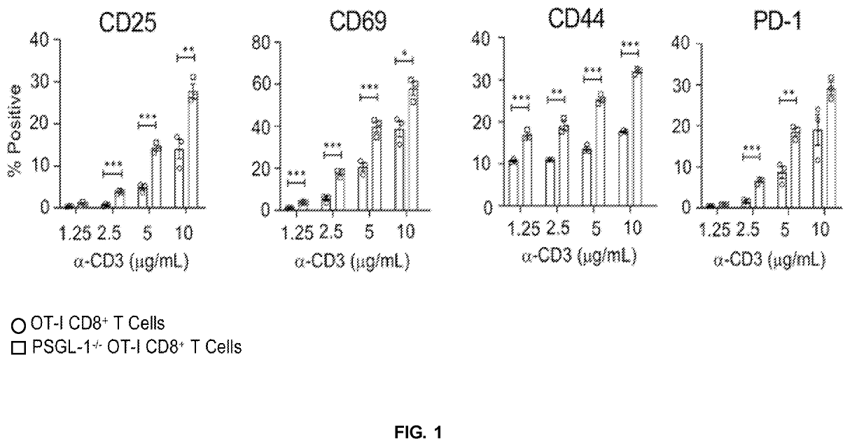

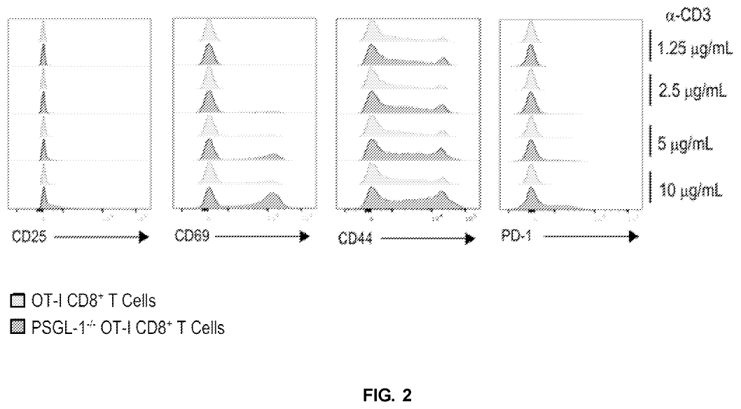

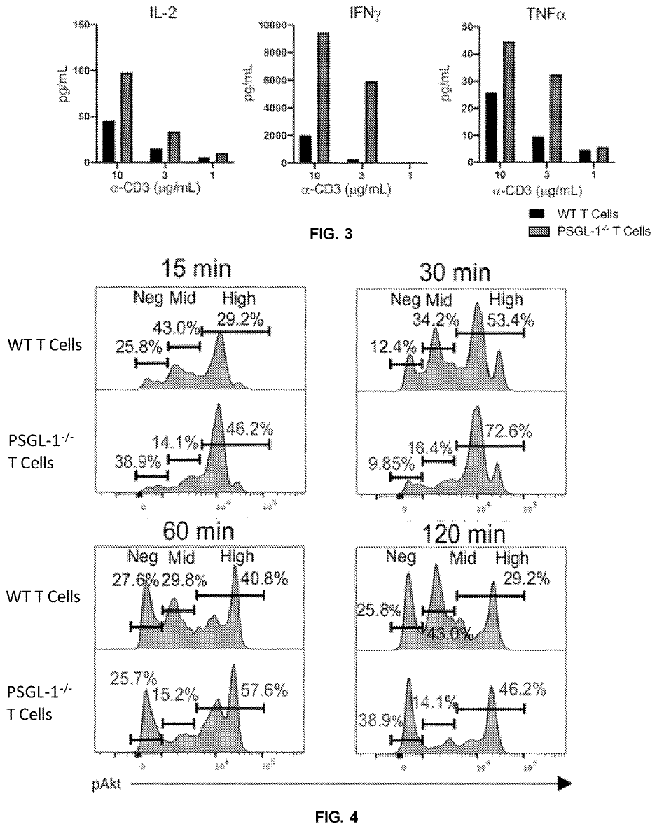

C57BL/6J wild-type (WT) and PSGL1 knockout (PSGL1−/−; B6·Cg-PSGL1tm1Fur/J) mice were backcrossed to C57B31.4L/6 Tg (TcraTcrb) 1100Mjb/J (OT-I) and B6·Cg-Tcratm1 (MomTg) (TcrLCMV) 327Sdz/TacMmjax (P14) to generate mice harboring either WT (PSGL1+/+; OT-I+; LCM+) or PSGL1 knockout (PSGL1−/−; OT-I+; LCMV+) CD8+ T-cells expressing T-cell receptors that recognize the a synthetic antigen comprising ovalbumin residues 257-264 SIINFEKL. Splenocytes were harvested from WT and PSGL1−/− mice of 6-8 weeks of age and pulsed with SIINFEKL OVA peptide. Activation of naïve WT and PSGL1−/−OT-I CD8+ T-cells following overnight treatment with an anti-CD3-epsilon antibody demonstrated that the absence of PSGL1 enabled greater activation as measured by expression of CD25, CD69, CD44, and PD1 (FIGS. 1-2) and enhanced cytokine production (FIG. 3).

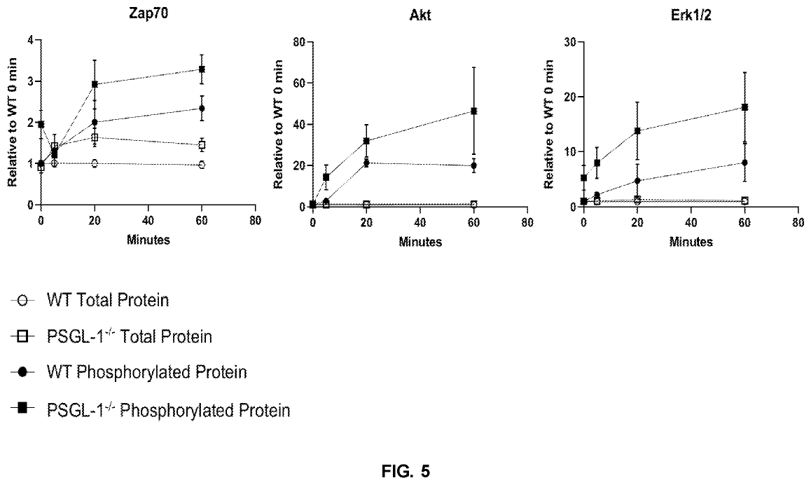

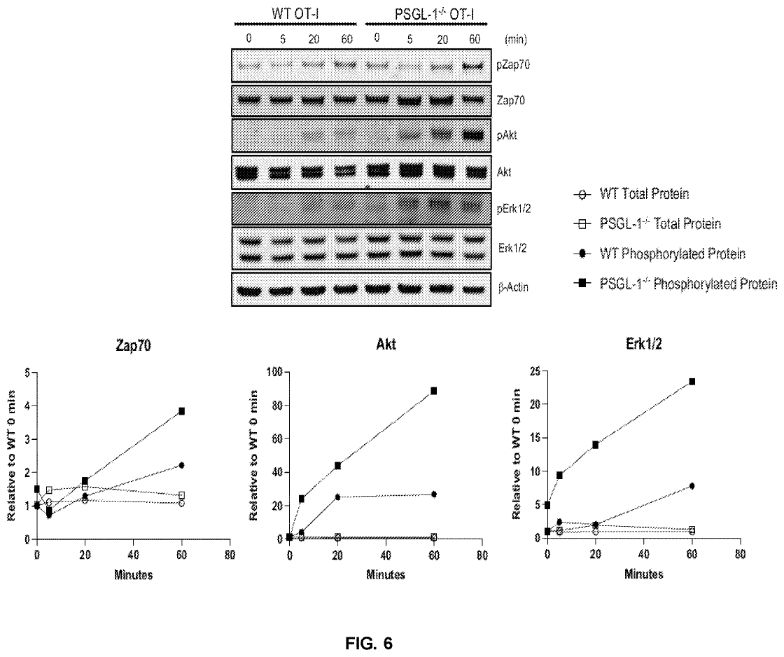

Akt phosphorylation, a direct biomarker of T-cell receptor signaling, was measured with flow cytometry and increased in PSGL1−/−OT-I CD8+ T-cells in comparison WT OT-I T-cells in that Akt phosphorylation levels were increased after 15 minutes and sustained for up to 2 hours (FIG. 4). Additionally, the kinetics of other biomarkers of TCR signaling were increased and sustained in PSGL1−/−OT-I T-cells in response to the anti-CD3-epsilon antibody treatment as measured by Western Blot, including phosphorylated Zap70 (pZap70), phosphorylated Erk1/2 (pERK), and phosphorylated Akt (FIG. 5-6). These data demonstrate that PSGL1 expression inherently limits T-cell activation and magnitude of the T-cell response in effector conditions (T_{EFF}) from the time of initial TCR engagement.

Bar graphs in FIG. 1 show frequencies of CD8+ T-cells expressing the indicated activation markers on OT-I WT and PSGL1−/−CD8+ T-cells following overnight (18 hr) stimulation with plate-bound anti-CD3-epsilon antibody at the indicated concentrations. Each dot represents an individual mouse. Data represent two independent experiments. P-values are as follows: *<0.05, **<0.01, ***<0.005. Data are normally distributed. Unpaired t tests were used for statistical analysis of parametric data. FIG. 2 depicts representative flow cytometry histograms of the data presented in FIG. 1.

FIG. 3 demonstrates that decreasing PSGL1 causes increased cytokine production in CD4+ and CD8+ T-cells in response to anti-CD3-epilson antibody stimulation for 3 days. Data are representative of experimental duplicate wells from one experiment.

Plots in FIG. 4 demonstrate that decreasing PSGL1 activity increased and prolonged Akt phosphorylation OT-I T-cells. Representative histograms showing expression levels of CD44 in WT and PSGL1−/−OT-I CD8+ T-cells in iT_{EFF} and iT_{EX} on day 5 post-activation.

In FIG. 5 the relative levels of phosphorylated Zap70, Erk1/2, and Akt following reduction of PSGL1 activity are demonstrated. Data are normalized to β-actin loading control and relative to WT samples at 0 minutes and averaged across three independent experiments with a pool of 2-3 mice per genotype per experiment.

FIG. 6 demonstrates that reduced PSGL1 signaling in T-cells is caused by increased and prolonged expression of pZap70, pErk1/2, and pAkt compared to WT T-cells following activation. Western blot detection of phosphorylated and total levels of Zap70, Erk1/2, AKT and GAPDH in WT and PSGL1−/−OT-I CD8+ T-cells stimulated for the indicated time with anti-CD3-epsilon antibody. Data are normalized to beta-actin and relative to baseline protein levels in WT samples at 0 minutes in the immunoblot. Data represents 3 independent experiments, 3 mice/genotype per experiment.

### Example 2: Reducing PSGL1 Increases the Sensitivity of T-Cell Receptor Signaling Under T-Cell Effector (T_{EFF}) or T-Cell Exhaustion (T_{EX}) Conditions

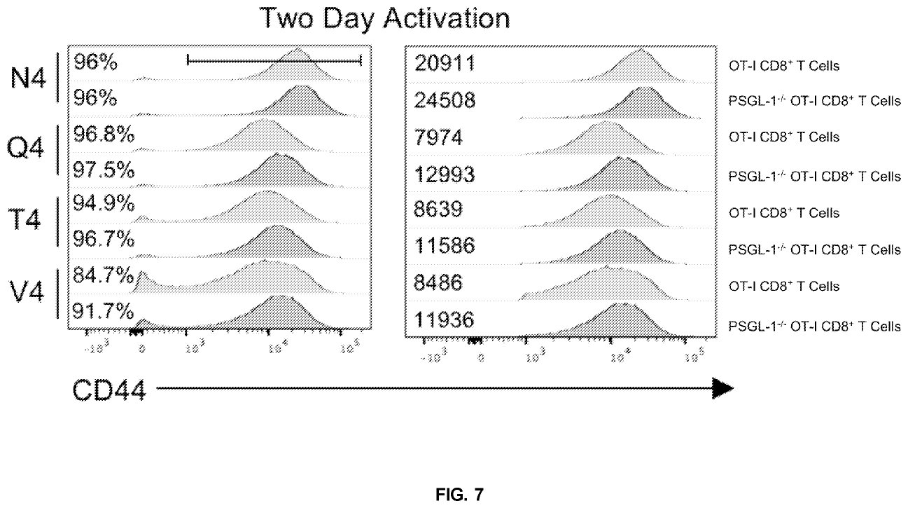

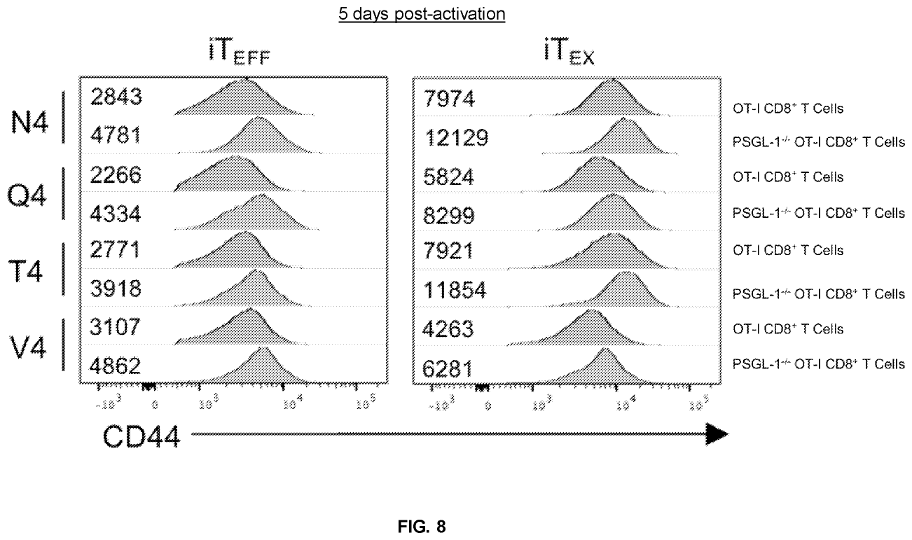

The effect of PSGL1 deficiency on TCR signaling sensitivity under conditions of induced T-cell exhaustion (iT_{EX}), WT and PSGL1−/−OT-I T-cells were repeatedly stimulated with variant, point-mutated OVA (257-264) peptides in vitro and were then compared to OT-I T_{EFF} conditions generated by optimal stimulation with a single peptide dose under induced T-cell effector conditions (iT_{EFF}). The variant OVA (257-264) peptides have different affinities for the OT-I TCR, which were used to determine whether PSGL1 deficiency could increase TCR sensitivity to lower-affinity ligands. iT_{EX} and rested iT_{EFF} were generated by stimulation with the SIINFEKL OVA (257-264) peptide (N4), or with the lower affinity variants, SIIQFEKL (Q4), SIITFEKL (T4), or SIIVFEKL (V4). Although equal frequencies of WT and PSGL1−/−OT-I T-cells were activated by N4, Q4, and T4 after a 2-day activation period, PSGL1−/−OT-I T-cells expressed higher levels of CD44 on a per-cell basis (MFI, median fluorescence intensity); notably, more PSGL1−/−OT-I T-cells were activated by the very low-affinity V4 peptide and expressed higher levels of CD44 than WT OT-I T-cells (FIG. 7). This pattern of CD44 expression was maintained under both iT_{EX} and rested-state iT_{EFF} after 5 days following the 2-day peptide exposure, when PSGL1−/−OT-IT-cells exhibited greater activation than WT OT-I T-cells stimulated with N4 peptide or the lower-affinity peptides (FIG. 8).

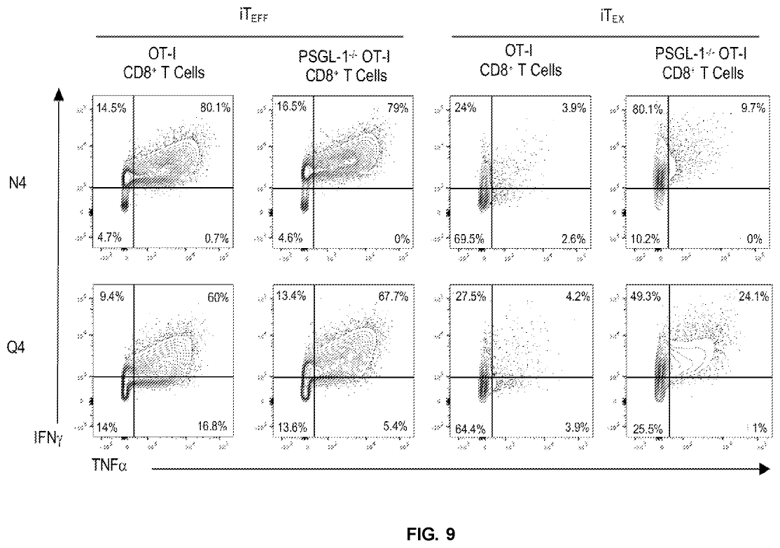

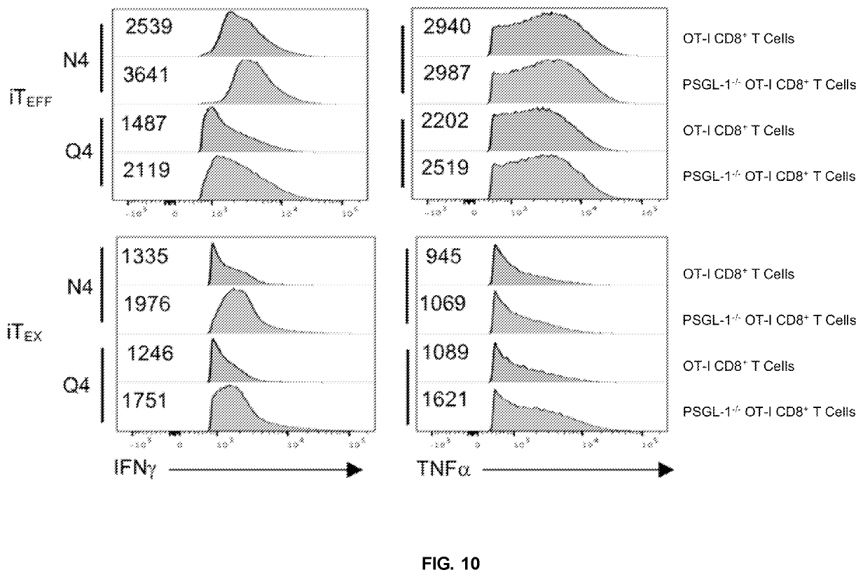

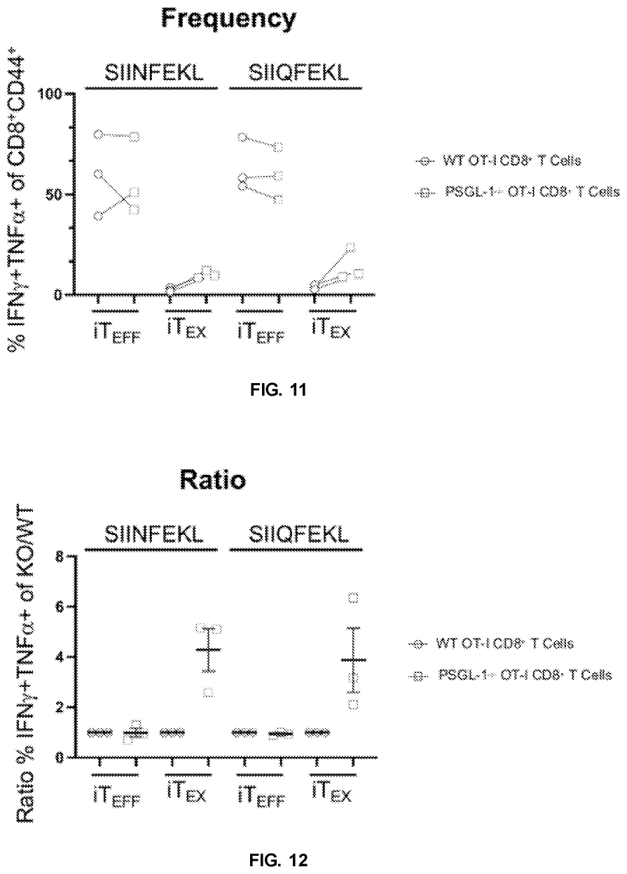

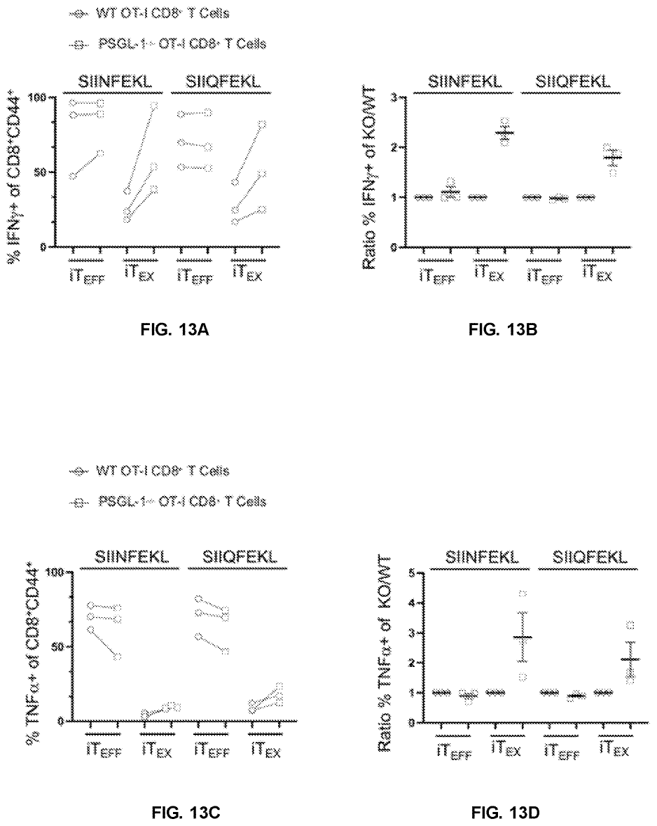

To assess the impact of affinity on T-cell function, WT and PSGL1−/−OT-I iT_{EFF} and iT_{EX} cells generated with the N4 or Q4 peptides were restimulated with the high-affinity N4 peptide to test for production of IFN-gamma and TNF-alpha (FIG. 9). WT and PSGL1−/−iT_{EFF} T-cells generated with either N4 or Q4 peptides had comparable frequencies of cytokine-producing T-cells, and most cytokine-producing T-cells produced both IFN-gamma and TNF-alpha as measured by immunofluorescent flow cytometry (FIG. 11, FIG. 12). However, PSGL1−/−iT_{EFF} T-cells demonstrated greater IFN-gamma production in comparison to WTiT_{EFF} T-cells (MFI) (FIG. 10). In contrast, WT iT_{EX} T-cells generated with the variant OVA (257-264) peptides were highly exhausted, with few cytokine-producing T-cells (FIG. 11, FIG. 12). However, PSGL1−/−iT_{EX} T-cells elicited with N4 stimulation exhibited a 4.2-fold increase in coproduction of IFN-gamma and TNF-alpha (FIG. 9 upper right, FIG. 11, FIG. 12) compared to WT iT_{EX} T-cells. Notably, PSGL1−/−CD8+ T-cells induced with the lower-affinity Q4 peptide retained an increased capacity for cytokine coproduction (FIG. 9 lower right, FIG. 11) that is increased by 3.8-fold over WTiT_{EX} T-cells (FIG. 12). In contrast, in WT and PSGL1−/−iT_{EFF} cultured with either N4 or Q4 peptide, the frequencies of IFN-gamma- or TNF-alpha-producing CD8+CD44+ T-cells remained equivalent (FIG. 13A, FIG. 13B). These results demonstrate that PSGL1 restrains early T-cell activation. Further, under conditions leading to exhaustion, PSGL1 limits responses to lower levels of TCR signals and lower affinity antigens, which would be detrimental to T-cell responses particularly in the context of lower-affinity tumor cell antigens.

FIG. 7 demonstrates that decreasing PSGL1 signaling increases TCR sensitivity and CD44 expression in OT-I T-cells 2 days after activation by N4, Q4, and T4 variant OVA peptides.

In FIG. 8, TCR sensitivity and CD44 expression are increased in OT-I T-cells when PSGL1 activity is reduced 5 days after activation by N4, Q4, and T4 peptides in both iT_{EX} and rested iT_{EFF} conditions.

FIG. 9 demonstrates that reducing PSGL1 activation increases IFN-gamma and TNF-alpha production in OT-I CD8+ T-cells. Representative FACS plots of IFN-gamma and TNF-alpha production by WT and PSGL1−/−OT-I CD8+ T-cells cultured with the indicated peptides under effector (left) or exhausted (right) conditions and restimulated on day 5 with SIINFEKL for 5 hours.

FIG. 10 shows representative histograms of IFN-gamma and TNF-alpha production by WT and PSGL1−/−OT-I CD8+ T-cells shown in FIG. 9.

FIG. 11 depicts a dot plot of the frequency of IFN-gamma and TNF-alpha double-producing CD44+OT-I CD8+ T-cells cultured under iT_{EFF} or iT_{EX} conditions following treatment either SIINFEKL or SIIQFEKL peptide for 5 days prior to 5-hour restimulation with the SIINFEKL peptide. Lines indicate the matched WT OT-I and PSGL1−/−OT-I CD8+ T-cells from the same experiment.

FIG. 12 depicts a dot plot of the frequency of the ratio of IFN-gamma and TNF-alpha double-producing PSGL1−/−OT-I to WT OT-I (KO/WT) CD44+OT-I CD8+ T-cells in iT_{EFF} or iT_{EX} conditions following treatment either SIINFEKL or SIIQFEKL peptide for 5 days prior to 5-hour restimulation with the SIINFEKL peptide. Lines indicate the matched WT OT-I and PSGL1−/−OT-I CD8+ T-cells from the same experiment.

FIG. 13 depicts dot plots of the frequencies of IFN-gamma- or TNF-alpha-producing WT and PSGL1−/−iT_{EFF} CD44+OT-I CD8+ T-cells cultured with either N4 or Q4 peptide (FIG. 13A, FIG. 13C). Dot plot of the frequency of the ratio of IFN-gamma or TNF-alpha single-producing PSGL1−/−OT-I to WT OT-I (KO/WT) CD44+OT-I CD8+ T-cells (FIG. 13B, FIG. 13D).

### Example 3: Reduced PSGL1 Signaling Changes TCR-Associated Proteomic Landscape in CD8+ T-Cells

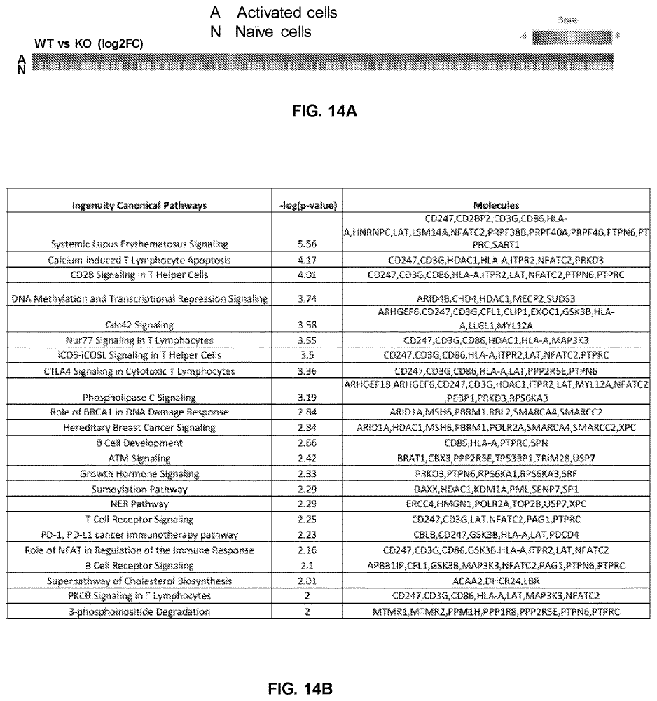

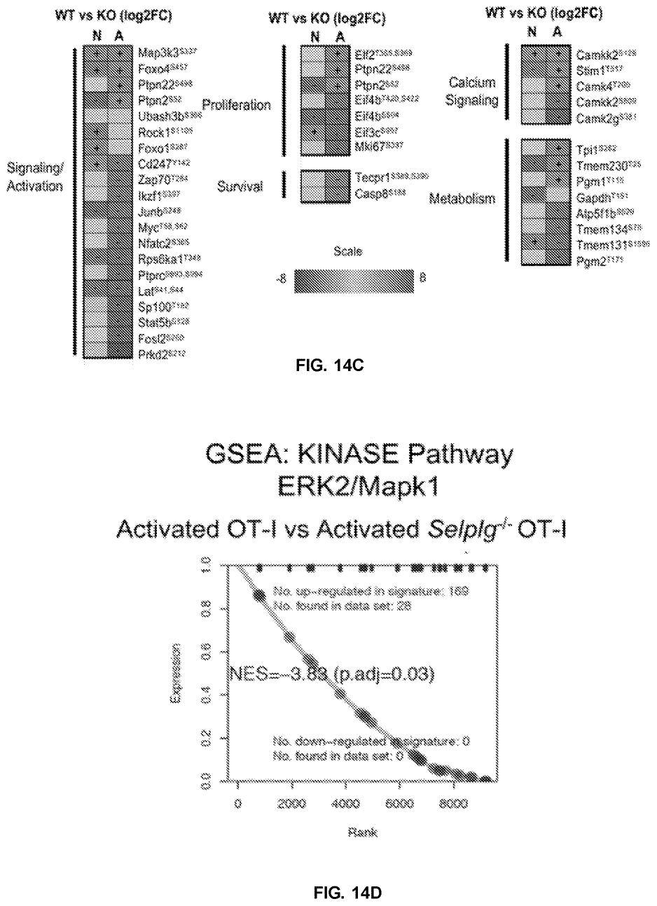

Multiplexed mass spectrometry and phospho-proteomics were performed on naïve WT and PSGL1−/−OT-I T-cells and also on anti-CD3-stimulated OT-I T-cells. Relative expression levels of 7,014 total and 9,294 phosphorylated proteins were evaluated. After activation with the anti-CD3 antibody, 576 phosphorylated proteins were differentially expressed between WT and PSGL1−/−OT-I T-cells (FIG. 14A). Pathway analysis of activated T-cells identified 23 enriched pathways, including “T cell receptor signaling” and “PD1/PD-L1 cancer immunotherapy” pathways (FIG. 14B). The phosphorylation state of proteins associated with regulation of T-cell signaling/activation were upregulated in PSGL1−/−OT-I T-cells compared to WT OT-I T-cells, including Zap70, Lat, Stat5b, Myc, Nfatc2, Junb, and Fosl (FIG. 14C, left). PSGL1 deletion also altered phosphorylation of proteins that regulate proliferation and survival, including Elf2, Ptpn22, Ptpn2, Eif4b, Eif3c, Mki67, Tecpr1, and Casp8 (FIG. 14C, middle); calcium signaling, including Camkk2, Stim1, Camk4, Camkk, and Camk2g (FIG. 14, right); and metabolism, including Tpil, Tmem230, Pgm1, Gapdh, Atp5f1b, Tmem134, Tmem131, and Pgm2 (FIG. 14C, right). Gene set enrichment analysis confirmed greater activation of the ERK1/2 pathway in PSGL1−/−OT-I cells (FIG. 14D). Few changes were observed in total protein expression in naïve WT cells compared to naïve PSGL1−/− cells (30 up, 14 down). Of the 576 differentially expressed phosphoproteins, only 22% were similarly differentially expressed in naïve T-cells.

FIG. 14A-14D further describe phospho-proteomics analysis, which reveals increased expression of molecules associated with T-cell activation in PSGL1 deficient CD8+ T-cells. FIG. 14A depicts a heat map of 576 phosphorylated proteins differentially expressed (≥2 log 2FC, FDR<0.01) between OT-I and PSGL1−/−OT-I CD8+ T-cells after 15 minutes of activation and the coordinate expression in non-activated, naïve cells (N). Data are displayed as expression in WT OT-I (WT) vs PSGL1−/−OT-I (KO) CD8+ T-cells. FIG. 14B shows a list of the 22 canonical pathways identified by IPA analysis with expression patterns potentially associated with activated WT OT-I vs PSGL1−/−OT-I CD8+ T-cells. FIG. 14C shows heat maps of selected genes and their expression in WT OT-I vs PSGL1−/−OT-I CD8+ T-cells at the naïve (N) state and after 15 minutes of activation in association with T-cell signaling/activation, proliferation and survival, and calcium signaling and metabolism. FIG. 14D depicts GSEA kinase pathway analysis of ERK2/Mapk1 in activated WT OT-I and PSGL1−/−OT-I CD8+ T-cells.

### Example 4: Glycosylation of PSGL1 Alters PSGL1 Localization Relative to TCR Signaling Complexes

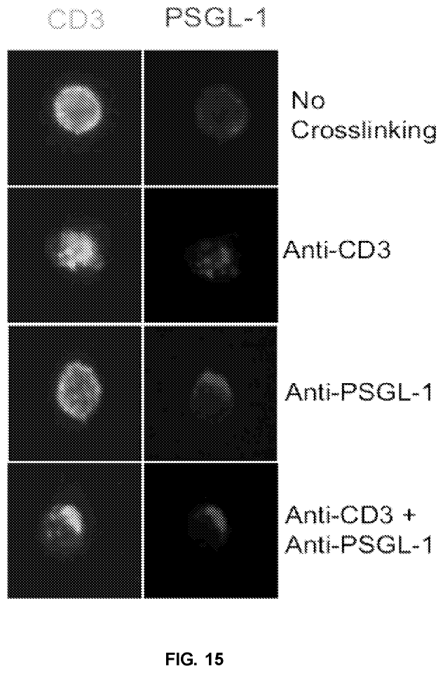

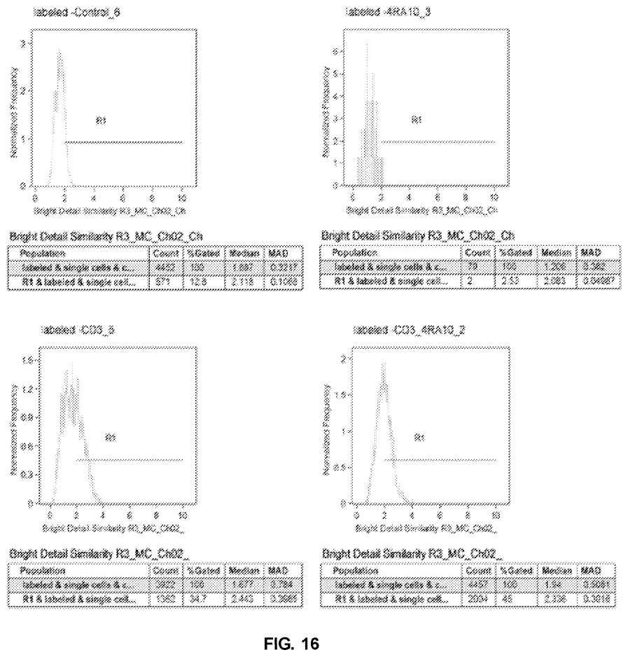

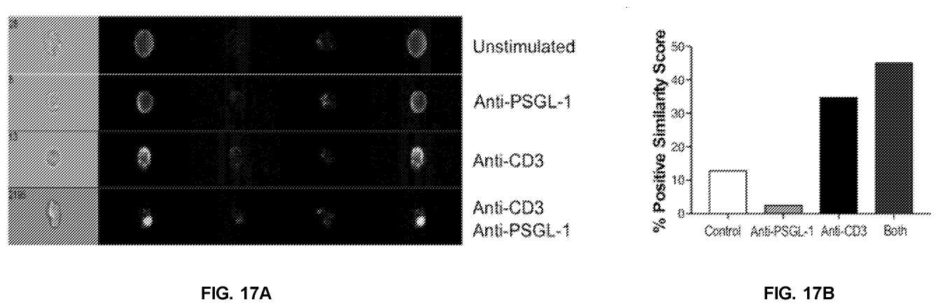

PSGL1 is a large, glycosylated protein that migrates to the uropod that forms at the back of T-cells during polarized movement. The spatial distribution of PSGL1 and the TCR complex on T-cells was analyzed by colocalization of PSGL1 and CD3-TCR complex with Amnis® imaging flow cytometry microscopy. On naïve T-cells, both PSGL1 and CD3 were evenly distributed over the cell (FIG. 15). Upon ligation with anti-CD3, CD3 formed punctate staining indicative of clustering. We observed PSGL1 co-clustering with CD3 under these conditions. Upon ligation with anti-PSGL1 alone, PSGL1 clustered to one side of the T-cell while CD3 remained dispersed. However, when both CD3 and PSGL1 were ligated simultaneously, CD3 and PSGL1 co-localized and migrated to one region of the cell. The positive similarity score determined by imaging flow cytometry of 10,000 CD8+ T cells (FIG. 16, FIG. 17A, FIG. 17B) confirmed and quantified the extent of CD3 and PSGL1 co-localization and show that PSGL1 is optimally positioned to directly regulate TCR signals particularly when PSGL1 is concomitantly engaged.

FIG. 15 further demonstrates that PSGL1 and TCR colocalize. Representative images (40×) of single OT-I CD8+ T-cells. Naïve OT-I CD8+ T-cells pre-stained with anti-CD3 and anti-PSGL1 antibodies were crosslinked as indicated to induce signaling by either CD3, PSGL1, or both and incubated for 10 minutes at 37° C. before fixation. Localization was detected using SA-FITC (green, CD3) or goat anti-rat IgG (red, PSGL1) and imaged on slides. Data represents 2 independent experiments. FIG. 16 further depict representative histograms from the Amnis ImageStream showing the % positive similarity score from immunofluorescent images in FIG. 15. Representative images of CD3 and PSGL1 localization using an Amnis ImageStream Imaging Flow Cytometer are further depicted in FIG. 17A. Imaging data from 10,000 individual cells was used to calculate the % positive similarity score shown, as shown in FIG. 17B, which represents data collected in 2 independent experiments.

### Example 5: Reduced PSGL1 Activity Decreases Biomarkers that Suppress TCR Signaling in CD8+ T-Cells

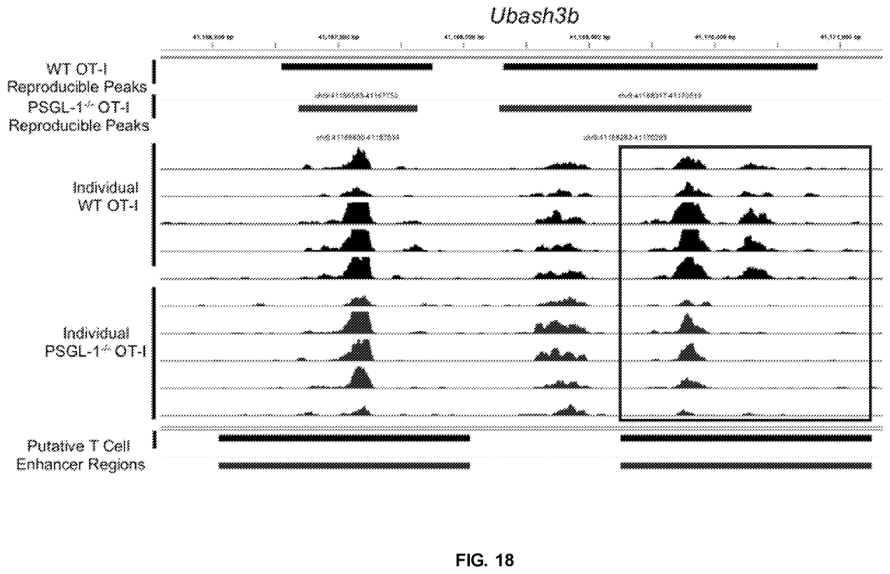

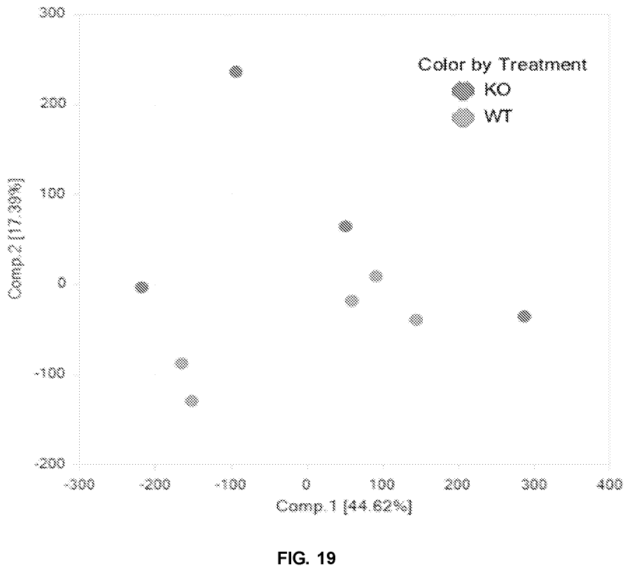

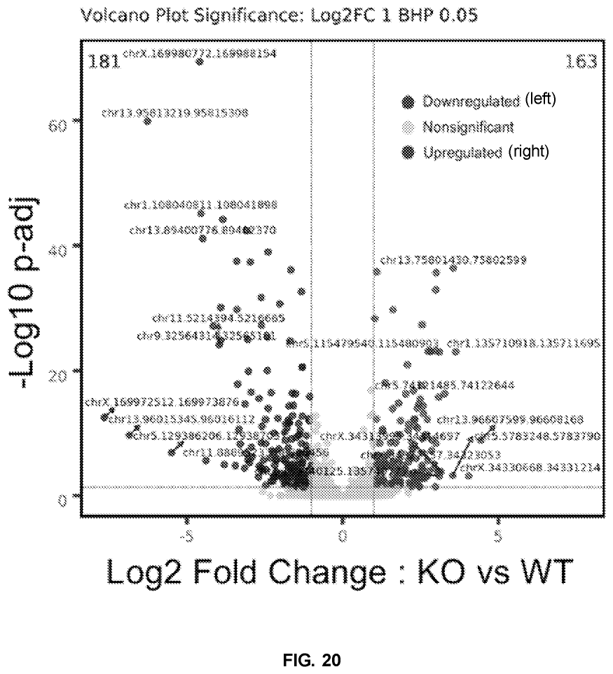

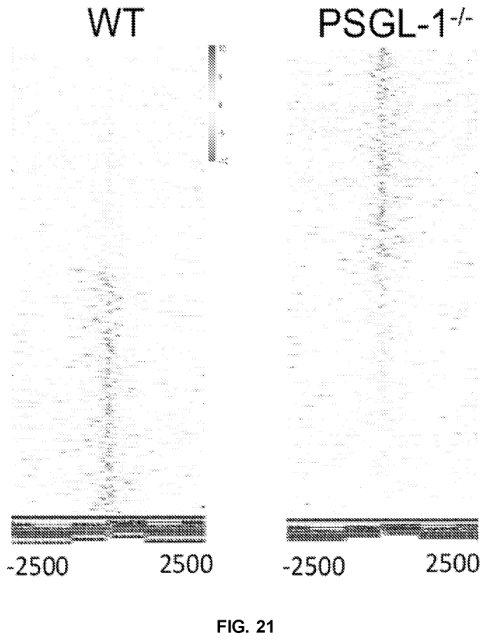

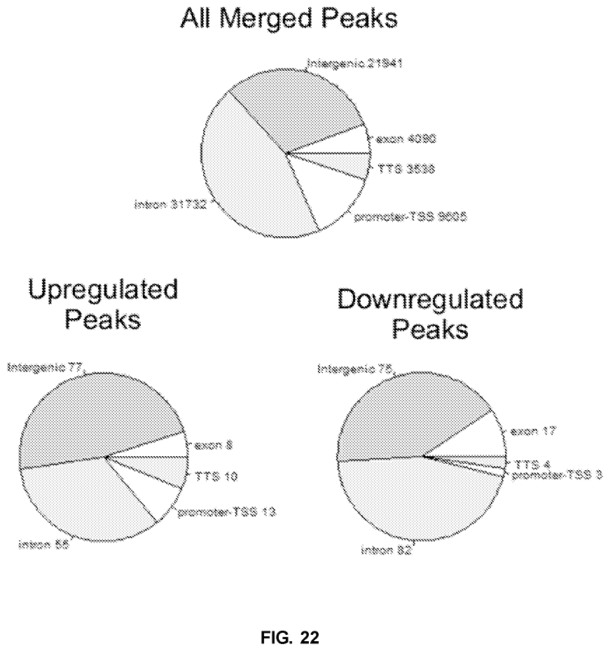

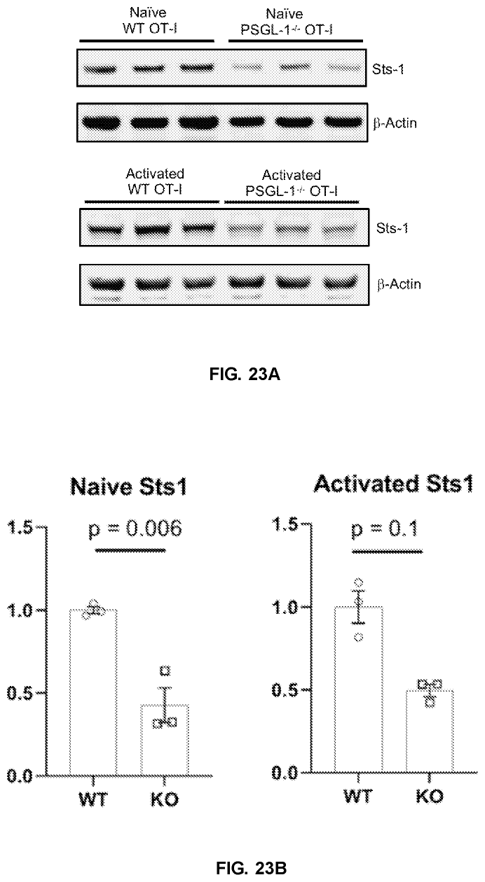

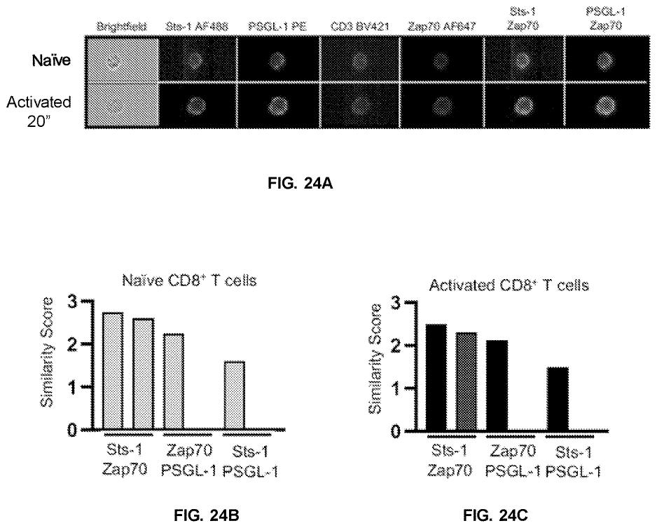

Analysis of LCMV virus-specific GP33-41 tetramer+CD8+ T-cells in WT and PSGL1−/− mice by RNA-seq 8 days post-LCMV C113 infection revealed a 3.25-fold downregulation of Ubash3b, which encodes the protein Sts-1 (Suppressor of T-cell signaling-1). Ubash3b expression negatively regulates TCR signaling by inhibiting Zap7020 in CD8+T_{EFF} cells and, consequently, limits the magnitude of T-cell activation. ATAC-seq (Assay for Transposase Accessible Chromatin Sequencing) tracings identified an enhancer region within Ubash3b with reduced open chromatin in PSGL1−/− T-cells compared to WT T-cells (FIG. 18, FIG. 19, FIG. 20, FIG. 21, FIG. 22). Protein analysis confirmed that naïve and activated PSGL1−/−OT-I T-cells express less Sts-1 (FIG. 23). Finally, imaging flow cytometry identified a high correlation in localization of Sts-1 and Zap70 in both WT and PSGL1−/−CD8+ T cells, as well as between PSGL1 and Zap70 (FIG. 24).

FIG. 18-FIG. 24 further demonstrate that reducing PSGL1 signaling decreases open chromatin within the Ubash3b gene. FIG. 18 depicts ATAC-seq (Assay for Transposase Accessible Chromatin Sequencing) tracings of chromatin accessibility within the Ubash3b gene region of WT OT-I tracks (black) and PSGL1−/−OT-I tracks (red). Each track represents biological replicates prepared from 2 separate experiments. FIG. 19 further depicts a principal component analysis (PCA) plot of individual WT (green) and PSGL1−/−(blue) ATAC-seq libraries.

FIG. 20 further depicts a volcano plot showing significantly upregulated (red), downregulated (blue), and unchanged chromatic accessibility in PSGL1−/−OT-I CD8+ T-cells compared to WTOT-I CD8+ T-cells based upon data acquired in ATAC-seq experiments. FIG. 21 further shows an ATAC-seq profile of reproducible chromatin accessibility in WT and PSGL1−/− libraries. FIG. 22 further depicts pie charts showing the breakdown by type in all, upregulated, and downregulated peaks determined by analysis of ATAC-seq data. FIG. 23 further demonstrates that reducing PSGL1 activity decreases Sts1 expression in both naïve and activated CD8+ T-cells. FIG. 23A further depicts a western blot analysis of Sts-1 and beta-Actin loading control protein expression in naïve (top) or two-day activated (bottom) WT and PSGL1−/−OT-I CD8+ T-cells. Each band is an independent biologic replicate. FIG. 23B depicts the quantification of Sts-1 protein expression normalized to beta-actin relative to the average WT Sts-1 levels in naïve (left) and activated (right) OT-I CD8+ T-cells of the blots shown in FIG. 23A.

FIG. 24A further depicts representative flow cytometry images of Sts-1, PSGL1, CD3, and Zap70 localization and co-localization in individual naïve (top row) or 20-minute-activated (bottom row) WT and PSGL1−/−OT-I CD8+ T-cells. Similarity scores of the indicated combinations of Sts-1, Zap70, and PSGL1 in naïve (FIG. 24B) or 20-minute-activated (FIG. 24C) WT (gray/black) and PSGL1 KO (light red, red) OT-I CD8+ T-cells as determined by Amnis Imaging Flow Cytometry. Imaging data from 10,000 individual cells was used to determine the similarity score. Representative of 2 independent experiments.

### Example 6: Reduced PSGL1 Signaling Increases Glycolysis in CD8+ T-Cells in T_{EFF} and T_{EX} Conditions

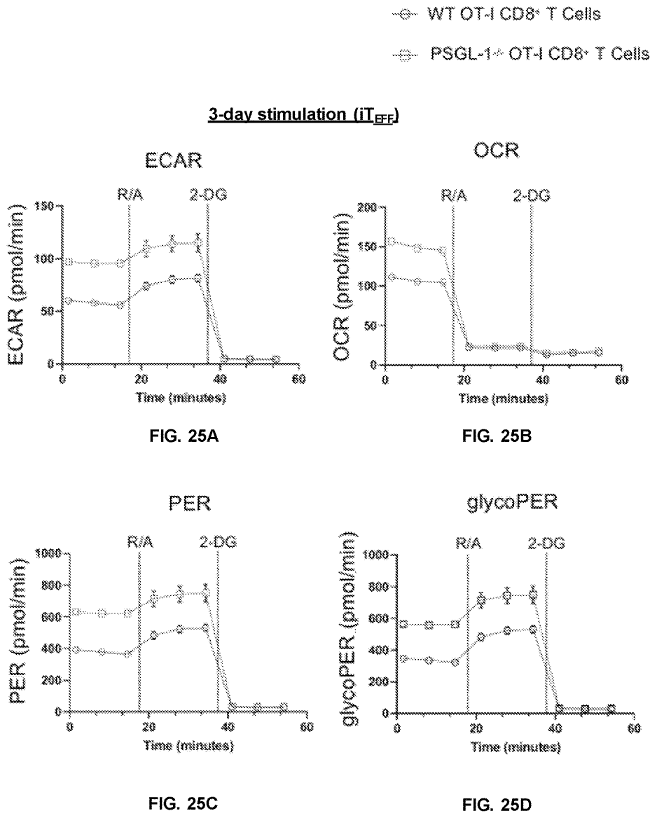

Glycolytic metabolism underlies T_{EFF} development upon TCR engagement, whereby increased glycolysis correlates with more robust activation of TCR signaling T-cells. Naïve WT or PSGL1−/−OT-I CD8+ T-cells were activated for 3 days in vitro with culture-plate-bound anti-CD3 and anti-CD28 in the presence of IL-2, and glycolytic metabolic parameters were measured as a proxy of T_{EFF} capacity. After the 3-day activation period, a Seahorse Glycolytic Rate assay with sequential addition of mitochondrial activity blockers rotenone A (R/A) and 2-deoxy-D-glucose (2-DG) was used to measure baseline and maximal glycolysis-related metabolic parameters in WT and PSGL1−/−OT-I T-cells. In comparison to activated WT OT-I cells, activated PSGL1−/− T-cells exhibited elevated baseline and maximal extracellular acidification rates (ECARs), oxygen consumption rates (OCRs), proton efflux rates (PER), and glycolytic rates (glycoPERs) (FIG. 25). These data demonstrate that PSGL1−/−OT-IT-cells have elevated glycolytic carbohydrate metabolism and greater capacity for T_{EFF}″ development upon TCR stimulation.

FIG. 25A-FIG. 25D further demonstrate that reduced PSGL1 signaling enhances glycolysis in CD8+ T-cells. Naïve WT or PSGL1−/−OT-I CD8+ T-cells were activated for 3 days with plate-bound anti-CD3 and anti-CD28 in the presence of IL-2. The extracellular acidification rate (ECAR, FIG. 25A) and the oxygen consumption rate (OCR, FIG. 25B) were assessed using the Seahorse Glycolytic Rate Assay at baseline and following the sequential addition of rotenone A and antimycin A (R/A), or 2-deoxy-D-glucose (2-DG) as indicated. The proton efflux rates (PER, FIG. 25C) and glycolytic rate (glycoPER, FIG. 25D) were calculated based on FIG. 25A and FIG. 25B. Data represent at least 3 individual experiments.

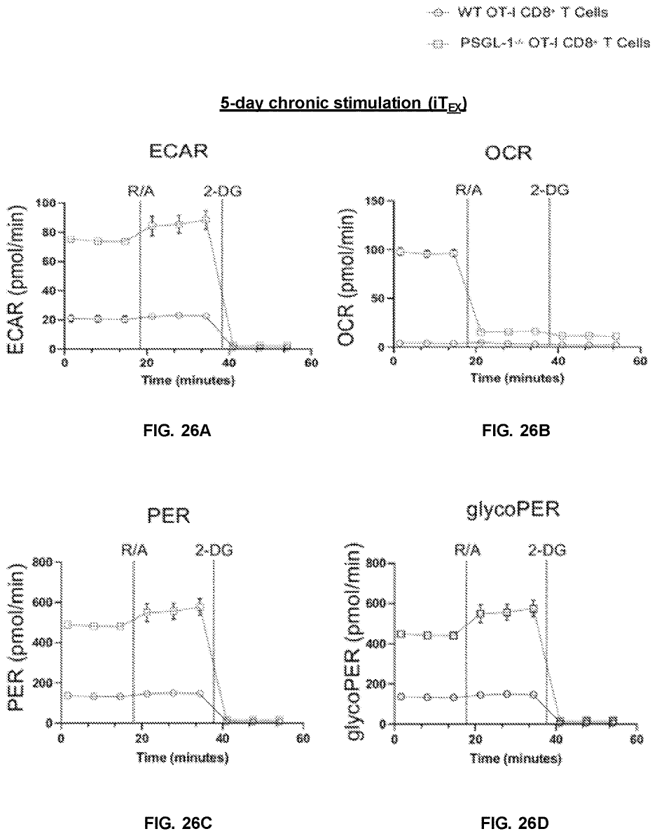

The effect of PSGL1 deficiency on the capacity of TCR signaling under T_{EX} conditions was also assessed. Naïve WT or PSGL1−/−OT-I CD8+ T-cells were activated for 5 days in vitro with repeatedly daily exposure to OVA257-264 peptide and culture-plate-bound anti-CD3 and anti-CD28 in the presence of IL-2 under T_{EX} conditions. After the 5-day activation period, the same Seahorse Glycolytic Rate assay (with sequential addition of R/A and 2-DG) was used to measure baseline and maximal glycolysis-related metabolic parameters in WT and PSGL1−/−OT-I T-cells. Exhausted WT OT-I iT_{EX} T-cells exhibited low levels of ECAR and OCR at baseline and upon mitochondrial uncoupling (FIG. 26A-FIG. 26D). In comparison to WT OT-I cells, PSGL1−/−OT-IiT_{EX} cells retained high baseline and maximal extracellular acidification rates (ECARs, FIG. 26A), oxygen consumption rates (OCRs, FIG. 26B), proton efflux rates (PER, FIG. 26C), and glycolytic rates (glycoPERs, FIG. 26D). These data indicate that PSGL1 deficiency increases glycolysis and capacity for T_{EFF} development in CD8+ T-cells under T_{EX} conditions of chronic TCR stimulation.

FIG. 26A-FIG. 26D further demonstrate that reduced PSGL1 signaling increases glycolysis in CD8+ T-cells under iT_{EX} conditions of chronic TCR stimulation. In vitro exhausted OT-I or PSGL1−/−OT-I CD8+ T-cells were generated as described in FIG. 25A-FIG. 25D and were subjected to a chronic 5-day stimulation of TCR signaling to induce iT_{EX} conditions. Glycolysis was assessed on day 5 using the Glycolytic Rate Assay. ECAR (FIG. 26A) and OCR (FIG. 26B) were measured at baseline and following the sequential addition of rotenone A and antimycin A, or 2-DG. PER (FIG. 26C) and glycoPER (FIG. 26D) were calculated based on FIG. 26A and FIG. 26B. Data represent at least 3 individual experiments.

### Example 7: Decreased PSGL1 Activity Enhances Glucose Uptake in CD8+ T-Cells Following Antigen Exposure

Increased glycolysis can be indicative of greater glucose utilization. A 2-NBD glucose (2-NBDG) uptake assay was performed using only solutions prepared in glucose-free, serum-free RPMI 1640 medium. Fluorescently labeled 2-NBDG (Cayman Chemical) uptake was performed with a 200 μM solution. Cells were incubated at 37° C. for 30 minutes before proceeding with fluorescence-activated cell sorting (FACS) staining. Cells were resuspended in FACS wash buffer, and 2-NBDG fluorescence intensity was analyzed with FACS within 30 minutes after 2-NBDG staining. The uptake of the fluorescent 2-NBDG was measured in WT and PSGL1−/−OT-I T-cells 2 hours after stimulation with SIINFEKL OVA257-264 peptide in vitro (FIG. 27A, FIG. 27B). No differences in glucose uptake by naïve (unstimulated) WT or PSGL1−/−OT-I T cells were observed (data not shown). PSGL1−/−OT-I T-cells exhibited a ~60% increase in 2-NBDG mean fluorescence intensity (MFI) compared to WTOT-I T-cells.

FIG. 27A and FIG. 27B further demonstrate that reduced PSGL1 activity increases glucose uptake in PSGL1−/−OT-I CD8+ T-cells. Representative histogram (FIG. 27A) and bar graph of quantified population average (FIG. 27B) of 2-NBDG uptake in OT-I or PSGL1−/−OT-I CD8+ T-cells after 2-hour stimulation with the SIINFEKL peptide. Each line/dot represents an individual mouse. Data are normally distributed.

### Example 8: Reduced PSGL1 Activity Enhances Glucose Uptake in CD8+ T-Cells in Tumor Microenvironments Ex Vivo

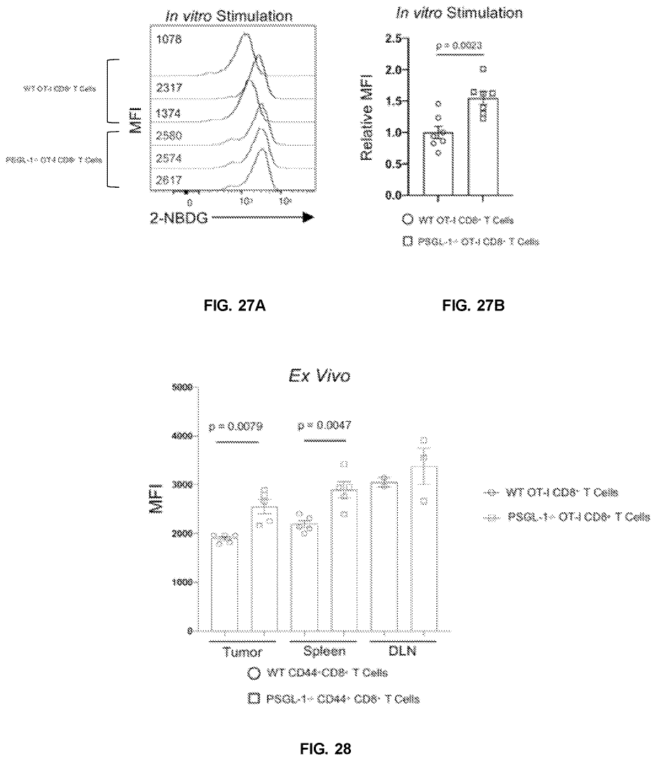

PSGL1−/− mice exhibit significant control of the YUMM1.5 melanoma tumor line, which is resistant to PD1 blockade. Differences in glucose uptake may contribute to an enhanced antitumor response. YUMM1.5 melanoma tumor-bearing WT and PSGL1−/−OT-I mice were sacrificed, and their tumors, spleens, and tumor-draining lymph nodes (DLNs) were harvested. A 2-NBDG uptake assay (see Example 7) was performed to measure glucose uptake in activated (CD44 hi) CD8+ T-cells obtained from the YUMM1.5-bearing mice, and MFI was measured by FACS. CD44 hi CD8+ T-cells from YUMM1.5 tumors, spleens, and tumor-draining lymph nodes (DLNs) were assessed immediately ex vivo. 2-NBDG uptake by PSGL1−/−CD44 hi CD8+ T-cells was elevated compared to WT CD 44 hi CD8+ T-cells in all 3 tested tissue samples (FIG. 28). Data are normally distributed except for WT tumors and DLN. Each dot in tumors and spleens represents an individual mouse; DLN represents a pool of mice. Data are representative of 1 of 2 independent experiments. The data show that PSGL1−/−CD8+ tumor-infiltrating T-cells (TILs) maintain a greater capacity for glucose uptake and enhanced glycolytic capabilities under exhaustive conditions.

FIG. 28 further demonstrates that reduced PSGL1 activity increases glucose (2-NBDG) uptake in melanoma tumor-infiltrating CD44+CD8+ T-cells. Graph of ex vivo 2-NBDG MFI values in CD44+CD8+ T-cells from tumors, spleens, or tumor draining lymph node (DLN) of WT or PSGL1 mice bearing YUMM1.5 tumors. Data are normally distributed except for WT tumors and DLN. Each data point in tumors and spleens groups represents an individual mouse; DLN data represents a pool of mice. Data represent 1 or 2 independent experiments.

### Example 9: Tumor-Infiltrated PSGL1-Deficient CD8+ T-Cells In Vivo have an Altered Gene Expression Profile

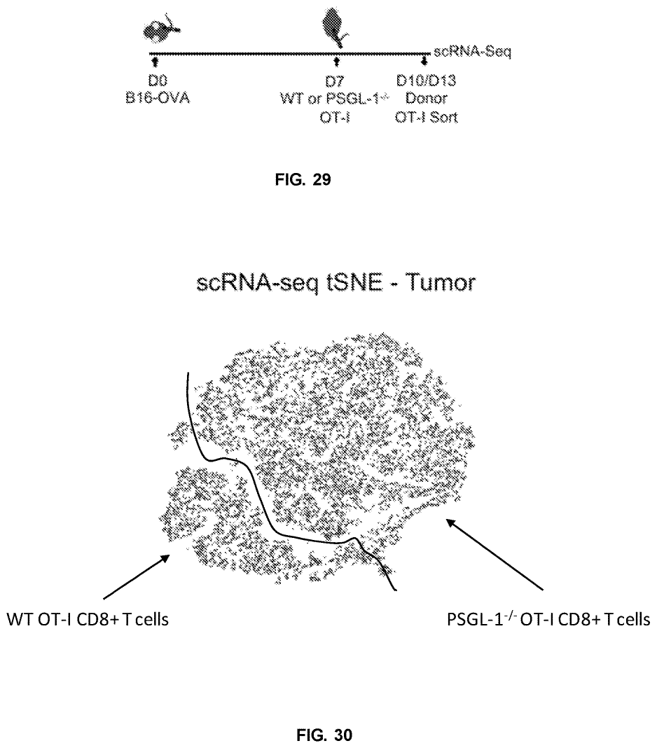

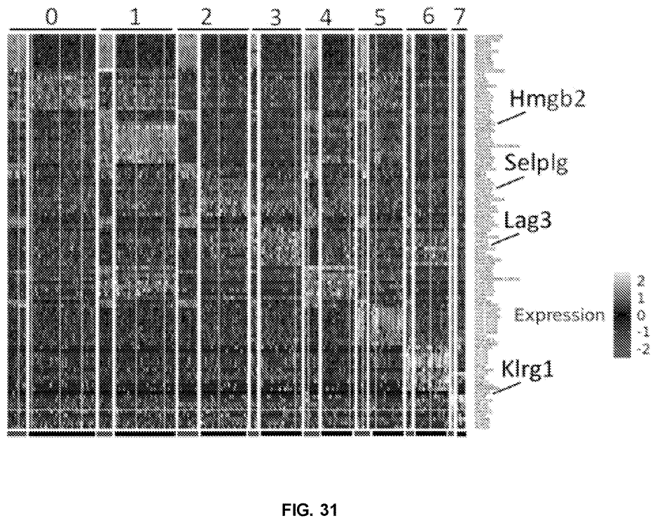

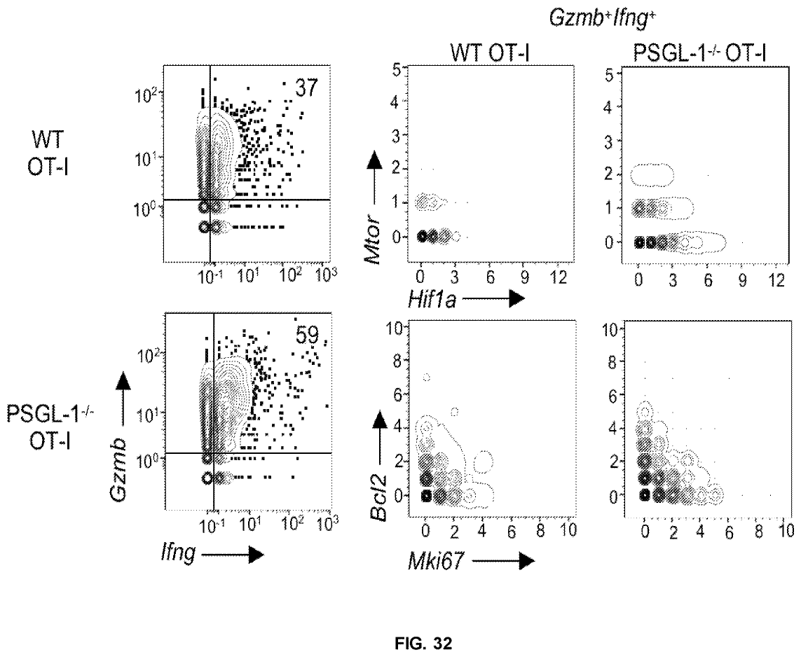

C57BL/6 (CD45.2/CD90.2) mice received a subcutaneous injection of 1×10⁶ B16-OVA melanoma cells. On day 7 mice received either 1×10⁶ in vitro activated WT OT-I (CD45.1+) or PSGL1−/−OT-I (CD90.1+) CD8+ T cells. Three days after T cell injection (day 10, overall), donor OT-I cells were FACS sorted from the inguinal tumor-draining and non-draining lymph nodes. Six days after T-cell injection (day 13 overall), donor OT-I T-cells were FACS sorted from the tumor-draining lymph nodes or B16-OVA tumors from a separate subset of mice. Experimental timeline is shown in FIG. 29. The B16-OVA mouse model was used in combination with single-cell RNA-sequencing (scRNA-seq) to identify transcriptional differences in donor WT or PSGL1−/−OT-I CD8+ T-cells (FIG. 29-FIG. 30). 10× Genomics single-cell RNA-sequencing (scRNA-seq) libraries were prepared immediately following FACS sorting and analyzed using CellLoupe (FIG. 30) and Seurat (FIG. 31). Our analysis identified genes that were globally and differentially expressed in PSGL1-deficient T-cells (e.g., Tmsb10, Crip1, Ifi2712a, Mt1, Mt2, Atp5k) as well as cluster-specific gene alterations (FIG. 31). SeqGeq (Flow Jo, BD) software allows the “gating” and evaluation of gene expression within a subset of scRNA-seq gene expression data and was used to identify OT-I cells co-expressing granzyme B (Gzmb) and IFN-gamma (Ifng) and to quantify gene expression within this subset of OT-I T-cells. In PSGL1−/−OT-I T-cells, co-expression of Gzmb and Ifng was linked to greater expression of Mtor and Hif1a as well as engagement in cell cycle (Mki67) (FIG. 32). No changes in cell survival-related genes were measured, exemplified by Bcl2 expression levels (FIG. 32).

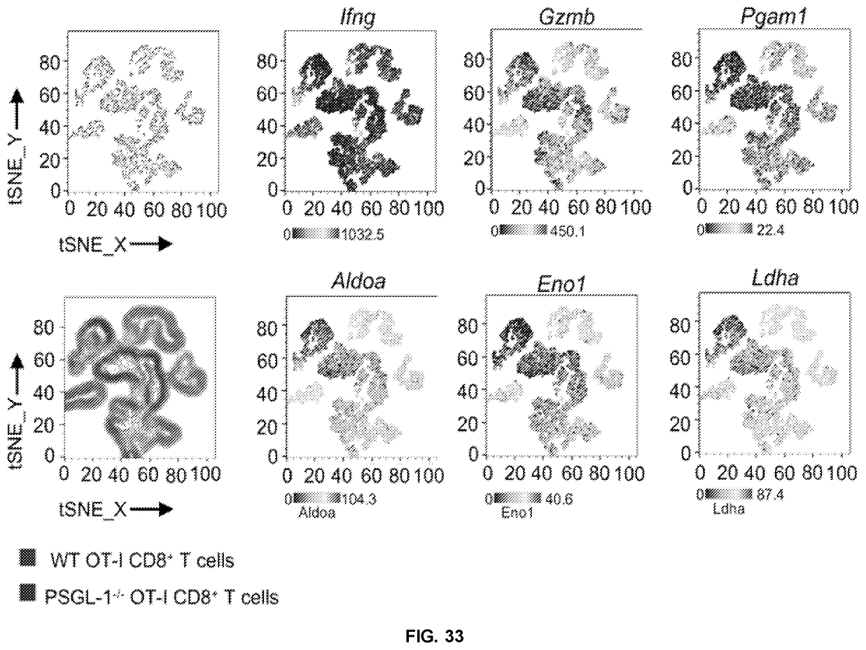

Expression of genes that regulate glycolysis was also quantified with scRNA-seq and SeqGeq for comparison between WT and PSGL1−/−OT-I T-cells. A SeqGeq-generated tSNE plot of overlaid WT (blue) and PSGL1−/−(red) OT-I T-cells gene expression profiles is shown in FIG. 33. Individual Ifng and Gzmb expression was largely localized in PSGL1−/−OT-I T cell clusters. Furthermore, greater expression levels of glycolysis-regulating genes (e.g., Pgam1, Aldoa, Eno1, and Ldha) were co-localized with the T_{EFF} PSGL1−/−OT-I T-cells. These data demonstrate that PSGL1 deficiency confers greater glycolytic capacity in the tumor microenvironment (TME) and that this greater metabolic capacity is linked to enhanced T_{EFF} function. Thus, PSGL1-deficiency releases metabolic constraints that are essential for efficient CD8+ T-cell effector function in the TME.

FIG. 30 further depicts a CellLoupe analysis of 10× Genomics single-cell RNA-sequencing (scRNA-seq) libraries of donor WT or donor PSGL1−/−OT-I CD8+ T-cells harvested from B16-OVA melanoma tumor-bearing mice prepared immediately following FACS sorting.

FIG. 31 further depicts a Seurat analysis of 10× Genomics single-cell RNA-sequencing (scRNA-seq) libraries of donor WT or donor PSGL1−/−OT-I CD8+ T-cells harvested from B16-OVA melanoma tumor-bearing mice prepared immediately following FACS sorting.

FIG. 32 further demonstrates that decreased PSGL1 signaling causes differential changes in cluster-specific gene expression in CD8+ T-cells. SeqGeq analysis of Gzmb and Ifng expression in WT and PSGL1−/−OT-I CD8+ T-cells (left). Top right: gene expression of Mtor and Hif1a in Gzmb+Ifng+WT and PSGL1−/−OT-I CD8+ T-cells. Bottom right: gene expression of Bc12 and Mki67 in Gzmb+Ifng+WT and PSGL1−/−OT-I CD8+ T-cells.

FIG. 33 further depicts a SeqGeq tSNE clustering analysis of WT (blue) and PSGL1−/−(red) OT-I CD8+ T-cells (top) with library source color overall (bottom). Gene expression overlays of Ifng, Gzmb, Pgam1, Aldoa, Eno1, and Ldha. All scRNA-seq data described herein and related to FIG. 29-FIG. 33 are representative of over 1,000 single cells per condition pooled from 6 mice per group.

### Example 10: Decreasing PSGL1 Signaling Increases the Generation of Stem Cell-Like T_{EFF} T-Cells During Chronic Viral Infection

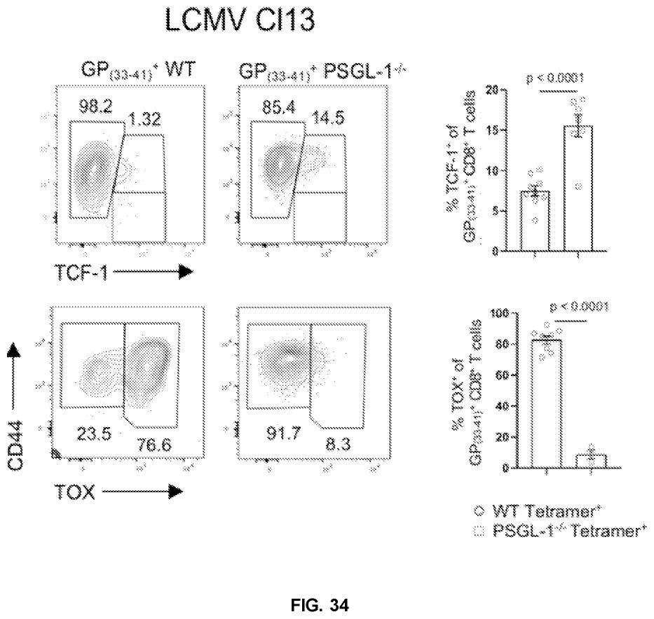

Studies of chronic Lymphocytic Choriomeningitis Virus (LCMV) infection have identified that stem-cell-like T-cells (TSC) with a capacity for self-renewal, expansion and effector function are defined by high expression levels of the transcription factor TCF-1. In metastatic melanoma, TCF-1+CD8+ T-cells in patients are associated with objective responses to PD1 blockade. The transcription factor, TOX, is a fundamental regulator that ultimately determines a T-cell's fate of irreversible exhaustion. However, co-expression of TOX and TCF-1 can distinguish less exhausted T-cells that can retain effector function. WT or PSGL1−/− mice were infected with LCMV C113 and virus-specific T-cells responses were assessed by flow cytometry. Expression levels of TCF-1 and TOX were measured in WT and PSGL1−/− virus-specific GP33-41+CD44 hi CD8+ T-cells and GP33-41+CD44-lacking CD8+ T-cells from the spleens of WT and PSGL1−/− mice at 15 days after infection (dpi), which is a time when the virus has cleared from the sera of PSGL1−/− mice (FIG. 34). Greater frequencies of TCF-1+ cells were found in samples prepared from PSGL1−/− mice compared to samples prepared from WT mice; conversely, a larger fraction of WT GP33-41+ T-cells expressed TOX in comparison to PSGL1−/−GP33-41+ T-cells.

FIG. 34 further demonstrates that decreasing PSGL1 signaling increases the generation of stem cell-like T_{EFF} T-cells during chronic viral infection. Representative flow cytometry plots showing TCF-1 or TOX expression vs CD44 expression in GP (33-41)-specific CD8+ T-cells from the spleens of WT or PSGL1−/− mice on day 15 after infection with LCMV CI13. Bar graphs represent frequencies of TCF-1+ (top) or TOX+ (bottom) GP (33-41)+CD8+ T cells in WT (circles) or PSGL1−/−(squares) mice on day 15 post-infection. Data are normally distributed. TCF-1 data are from 2 independent experiments, TOX data are from one experiment. Each data point represents an individual mouse.

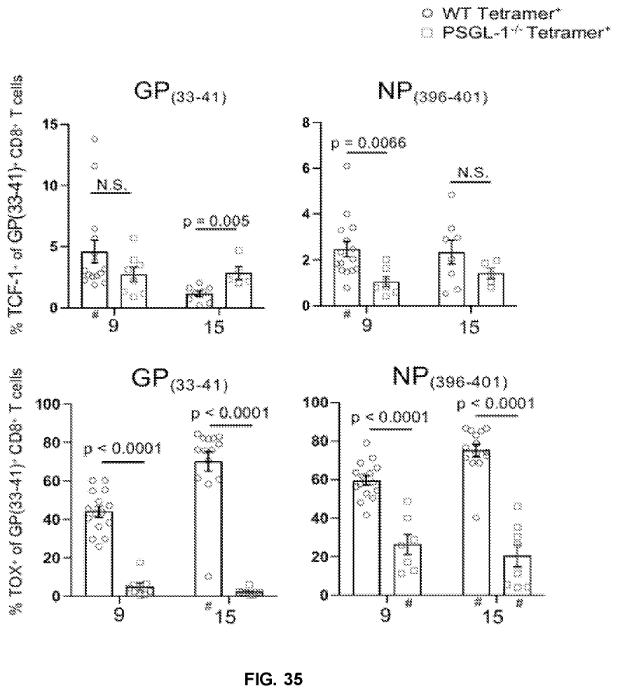

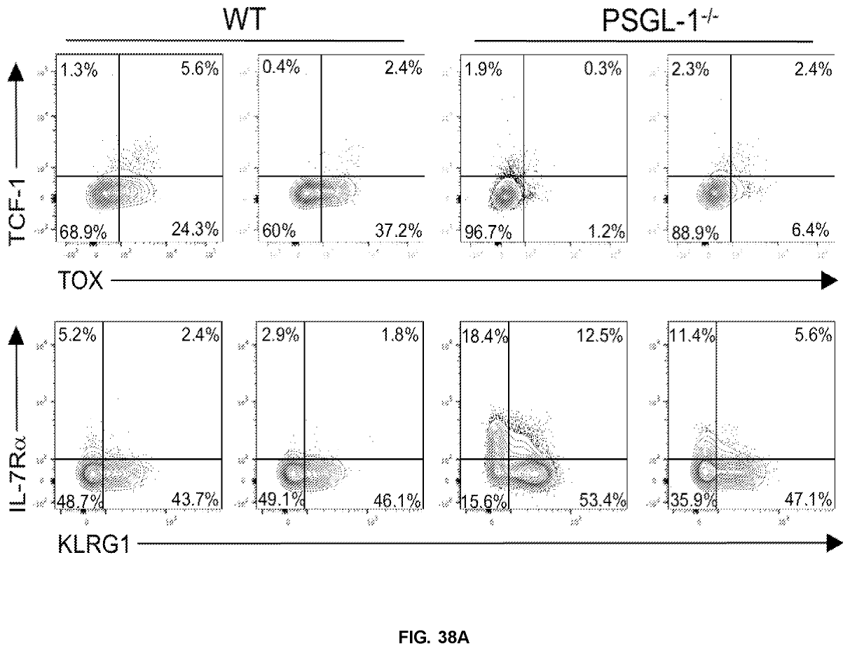

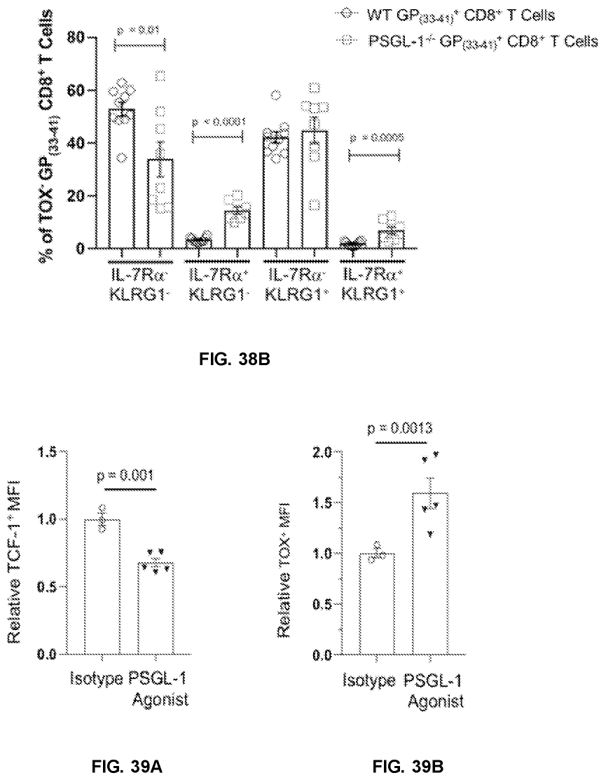

Expression levels of TCF-1 and TOX were also measured in virus-specific GP33-41+ and NP396-404+ T-cells within WT and PSGL1−/− mice at 9 dpi, which is a time when the virus loads are comparable. The frequencies of TCF-1+ T-cells in the blood were indistinguishable between WT and PSGL1−/− mice, but dramatically fewer GP33-41+ T-cells from PSGL1−/− mice expressed TOX (FIG. 35). At 15 dpi, the phenotype of GP33-41+ T-cells from PSGL1−/− mice showed a moderate increase in TCF-1+ T-cells and also a drastic decrease in TOX+ cells (FIG. 35). In both WT and PSGL1−/− tissue samples, the majority of GP33-41+ T-cells co-expressed TCF-1− and TOX− (FIG. 35, FIG. 38).

FIG. 35 further demonstrates that decreased PSGL1 signaling robustly decreases TOX expression T-cells during chronic viral infection. Frequencies of TCF-1+ (top) or TOX+ (bottom) GP (33-41)+ or NP (396-404)+CD8+ T-cells in the blood of WT (circles) or PSGL1−/−(squares) mice on days 9 and 15 post-infection (dpi). Data are normally distributed except those noted by #. Data are from 3 independent experiments. Each data point represents an individual mouse.

### Example 11: Decreasing PSGL1 Activity Prevents Terminal Differentiation of Exhausted T-Cells

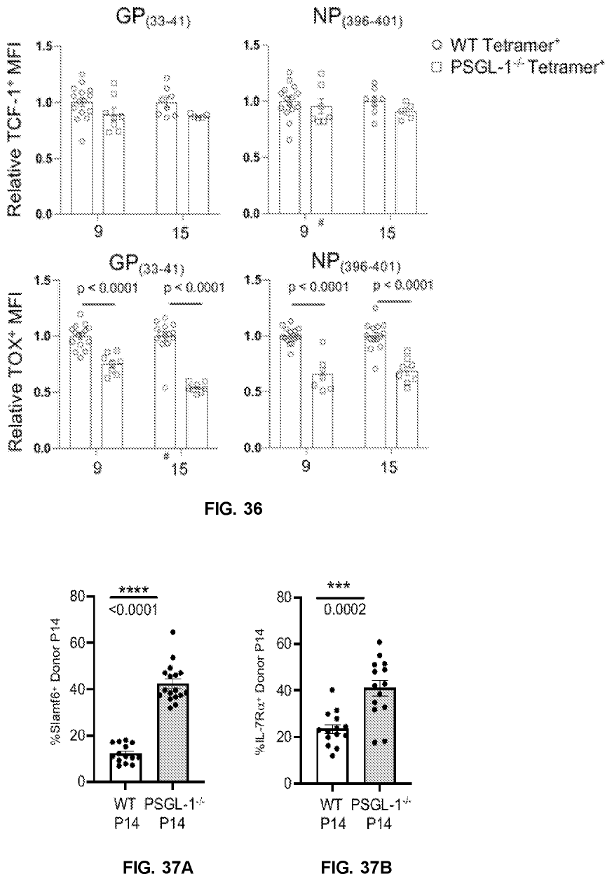

Unlike GP33-41+ T cells, which persist throughout LCMV C113 infection, CD8+ T-cells recognizing the higher-avidity epitope NP396-404 are depleted. However, NP396-404 CD8+ T-cells are retained in PSGL1−/− mice. WT and PSGL1−/− mice were infected with LCMV CI13, and their blood was collected on days 9 and 15 post-infection, upon which CD8+ T-cells were isolated and analyzed by FACS; the frequencies (i.e. represented populations) of TCF-1+ or TOX+GP (33-41)+ or NP (396-404)+CD8+ T cells were then analyzed. Within NP396-404+ T-cells, fewer PSGL1−/−T-cells expressed TCF-1 than WT T-cells at 9 dpi, while populations of TCF-1+NP396-404+ T-cells were equally represented in WT and PSGL1−/− tissue at 15 dpi. However, lower frequencies of PSGL1−/− T-cells expressed TOX at both 9 dpi and 15 dpi were detected compared to WT T-cell populations (FIG. 35). Further, while expression levels of TCF-1 were indistinguishable between GP33-41+ and NP396-401+ T-cells, TOX expression was consistently lower in PSGL1−/− virus-specific T-cells (FIG. 36). These data indicate that PSGL1 deficiency prevents terminal differentiation of exhausted T-cells (T_{EX}), in part, by limiting the expression of TOX. To confirm that this is an intrinsic cellular effect, equal numbers of WT P14 and PSGL1−/−P14 CD8+ T-cells were transferred into LMCV CI13-infected WT mice, and donor T-cells in the blood were collected and analyzed at 60 dpi. WT mice received a 1:1 mix of WT P14 and PSGL1−/−P14 (5×10⁵ cells each) followed by infection with LCMV C113, and Slamf6 and IL-7R-alpha expression on donor T-cells in the blood were assessed by flow cytometry on day 60 post-infection. While the frequency of donor WT and PSGL1−/− were comparable at this timepoint (60 dpi), more PSGL1−/−P14 CD8+ T-cells displayed IL-7R-alpha and Slamf6+, the latter of which is highly co-expressed with TCF-1 on progenitor TSC (FIG. 37). These data indicate that PSGL1 signaling intrinsically regulates CD8+ T-cell differentiation in response to repeated antigen exposure.

FIG. 36 further demonstrates that decreased PSGL1 signaling consistently decreases TOX expression in CD8+ T-cells during chronic viral infection. The relative per-cell expression (MFI) of TCF-1+ or TOX+GP (33-41)+ or NP (396-404)+CD8+ T-cells in the blood of WT (circles) or PSGL1−/−(squares) mice on days 9 and 15 post-infection. Data are normally distributed except those noted by #. Data are from 3 independent experiments. Each data point represents an individual mouse.

FIG. 37A and FIG. 37B further demonstrate that decreased PSGL1 signaling increases the prevalence of Slamf6+ and IL-7R-alpha+ stem-cell-like T-cells (TSC). Frequency of Slamf6+ and IL-7R-alpha+WTP14 (white bars) and PSGL1−/−P14 (gray bars) donor T-cells in the blood of LCMV CI13-infected WT mice assessed by flow cytometry on day 60 post-infection. 5×10⁵ donor T-cells administered per mouse.

### Example 12: Reduced PSGL1 Activation Increases Prevalence of “Long-Lived” CD8+ T-Cells During Viral Infection

Differential IL-7R-alpha and KLRG-1 expression by CD8+ T-cells classically delineates short-lived effector cells (SLEC) and memory precursor cells (MPEC) with some infections and with LCMV Arm infection, and GP33-41-specific PSGL1−/−CD8+ T-cells are skewed towards the MPEC phenotype. Previous studies have also demonstrated an inverse relationship between TOX and KLRG1 expression during LCMV C113 infection. Here, KLRG1+ T-cells were within the TOX-T-cell population in both WT and PSGL1−/−GP33-41+ T-cells (data not shown). The differentiation states of TOX-GP33-41+ T-cells were evaluated, which revealed that there were fewer IL-7R-alpha/KLRG-1-PSGL1−/−CD8+ T-cells and an increased representation of IL-7R-alpha/KLRG-1− and IL-7R-alpha/KLRG-1+ T-cell populations (FIG. 38).

FIG. 38A and FIG. 38B further demonstrate that reduced PSGL1 activity increased the prevalence of memory precursor cells despite viral infection. FIG. 38A (top row) further depicts representative flow cytometry plots of TCF-1 and TOX expression in GP (33-41)-specific CD8+ T-cells from the blood of WT or PSGL1−/− mice at 9 days post-infection (dpi). Bottom row of panels in FIG. 38A further depict representative flow cytometry plots of IL-7R-alpha and KLRG1 expression in TOX-GP (33-41)-specific CD8+ T-cells from the blood of WT or PSGL1−/− mice at 9 dpi. Bar graphs in FIG. 38B further depicts the frequency of IL-7R-alpha and KLRG1 expression in GP (33-41)+CD8+ T-cells in the blood of WT (circles) or PSGL1−/−(squares) mice at 9 dpi. Data are normally distributed (measured by Shapiro-Wilk test). An unpaired t-test was used for statistical analysis. Data represent 1-2 independent experiments. Each data point represents an individual mouse.

### Example 13: Pharmacological PSGL1 Agonist Drives T_{EX} Differentiation and Reduces T_{EFF} Differentiation

To assess whether PSGL1 engagement is a driver of TOX and T_{EX} differentiation, ex vivo TCF-1 and TOX expression levels were quantified in virus-specific GP33-41+ T-cells obtained from LCMV C113-infected WT mice that were treated with either an agonist anti-PSGL1 antibody (full-length clone 4RA 10, “4RA10-FL”) or an isotype control antibody. WT mice were infected with LCMV C113 and were treated with either isotype control antibody or PSGL1 antibody (4RA10-FL) on days 5 dpi and 8 dpi. At 15 dpi, spleens were harvested and CD8+ T-cells were subjected to immunofluorescent FACS, and the relative per-cell expression (MFI) of TCF-1+ or TOX+GP (33-41)+ or NP (396-404)+CD8+ T-cells was quantified. PSGL1 ligation caused by 4RA10-FL treatment during LCMV C113 infection concomitantly reduced the expression of TCF-1 by 25% and increased TOX expression by 50% (FIG. 39).

FIG. 39A and FIG. 39B further demonstrate that increasing PSGL1 activity decreases T_{EFF} biomarkers and increases TOX. On day 15 dpi, relative per-cell expression (MFI) of TCF-1+ or TOX+GP (33-41)+ or NP (396-404)+CD8+ T-cells in the spleens of LCMV C113-infected WT mice treated with either isotype control antibody or agonist PSGL1 antibody (4RA10) on days 5 and 8 dpi. Data are normally distributed and represent 2 independent experiments. Each data point represents an individual mouse.

### Example 14: Reduced PSGL1 Activity Increases T_{EFF} Biomarkers, Reduces T_{EX} Biomarkers in CD8+ T-Cells in a Mouse Model of Human Melanoma

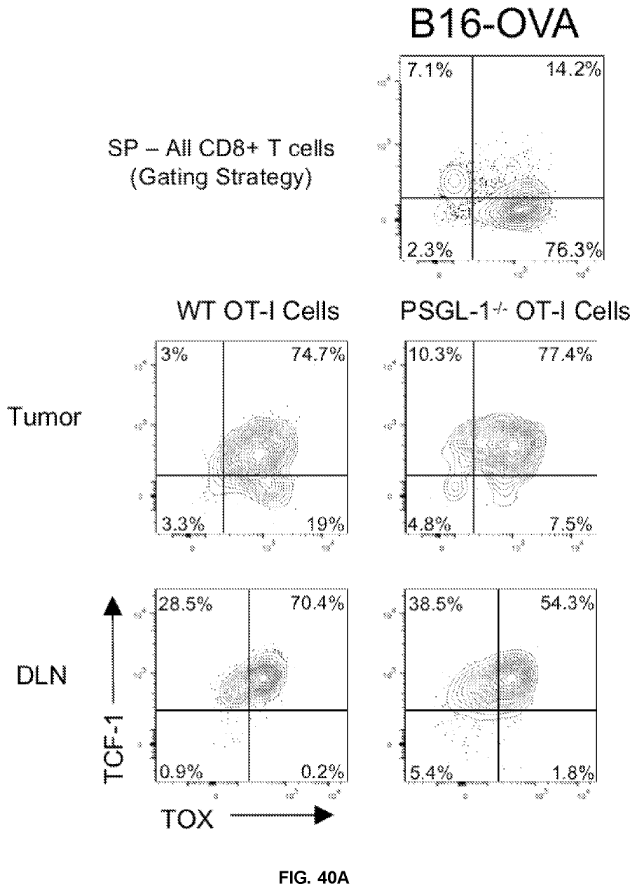

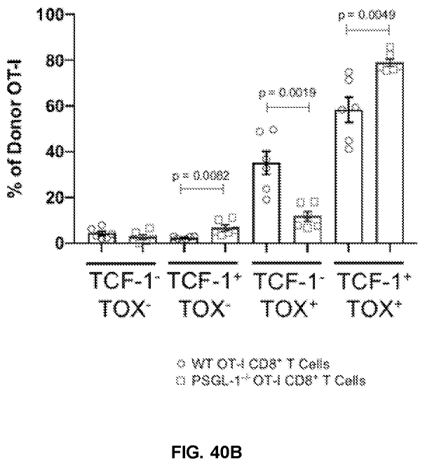

To address T cell intrinsic effects of PSGL1 regulation on TCF-1 and TOX expression in response to melanoma tumors, adoptively transferred activated CD44 hi WT and PSGL1−/−OT-I T-cells were collected and analyzed by FACS 10 days after transfer into B16-OVA tumor-bearing WT mice. WT (CD45.2/CD90.2) mice were inoculated subcutaneously with 1×10⁶ B16-OVA melanoma cells. Two weeks later, mice received either 1×10⁶ in vitro-activated WT OT-I (CD45.1+) or PSGL1−/−OT-I (CD90.1+) CD8+ T-cells. Six days later, donor OT-I T-cells in B16-OVA tumors or inguinal tumor-draining lymph nodes (DLNs) were analyzed. A modest increase in the frequencies of TCF-1+CD44+OT-I+ T-cells in B16-OVA tumors obtained from PSGL1−/− mice was observed in comparison to tumors obtained from WT mice (FIG. 40A, middle panels). In the tumor-draining lymph node, essentially all donor OT-I T-cells were TCF-1+, but fewer T-cells co-expressed TOX in PSGL1−/−OT-I T-cells (FIG. 40A, bottom panels). Evaluation of TCF-1 and TOX showed that PSGL1−/−OT-I TILs displayed a modest increase in TCF-1+ single-positive (SP) cells, but significantly decreased TOX+SP cells and increased TCF-1/TOX double-positive (DP) cells compared to WTOT-I cells (FIG. 40B). These data suggest that a substantial proportion of tumor-specific T_{EX} cells were progressively differentiating during the response within the tumors. In addition, decreased TOX expression consistently distinguished PSGL1−/− T-cells in a cancerous tumor microenvironment, which demonstrates a fundamental role for PSGL1 in promoting T-cell exhaustion.

FIG. 40A-FIG. 40B further demonstrate that decreased PSGL1 activity increases the prevalence of activated T_{EFF} CD8+ T-cells. FIG. 40A depicts TCF-1 and TOX expression in CD44 hi donor OT-I CD8+ T-cells by flow cytometry. T-cells were obtained from tumors or inguinal tumor-draining lymph nodes of WT mice inoculated with B16-OVA melanoma cells. FIG. 40B depicts population mean quantification of TCF-1 and TOX expression in donor OT-I CD8+ T-cells in the tumors of B16-OVA tumor-bearing mice. Data are normally distributed and represent 2 independent experiments. Each data point represents an individual mouse.

### Example 15: PSGL1 Activity is Robustly Elevated in CD8+ T-Cells of Melanoma Patients

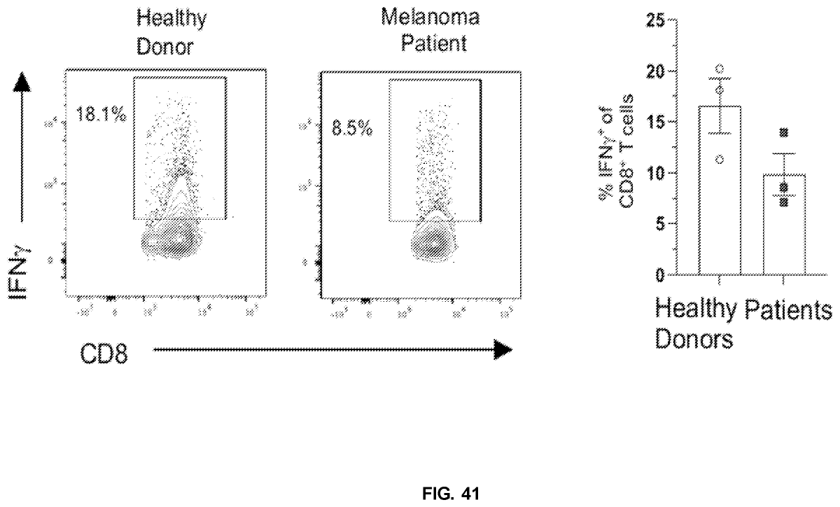

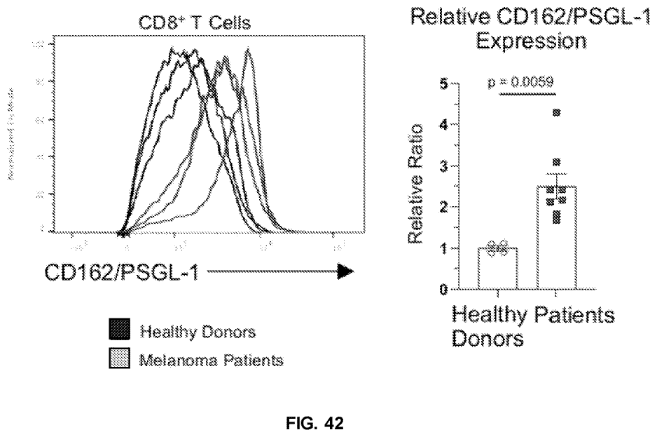

Cancerous tumor environments exhaust T-cells and reduces their T_{EFF} response. PSGL1 promotes T_{EX} differentiation and suppresses T_{EFF} differentiation. In melanoma patients, CD8+ T-cells have increased expression levels of PD1 and CTLA-4. Thus, PSGL1 expression was expected to be increased on CD8+ T-cells obtained from melanoma patients, similarly to PD1 or CTLA-4 expression. PBMCs from healthy donors (n=4) or melanoma patients (n=8) were cultured for 2 days with anti-CD3 and anti-CD28. PSGL1 (CD 162) expression and cytokine production were evaluated on PBMCs from healthy donors and from melanoma patients after stimulation with anti-CD3/CD28, CD8+ T-cells from melanoma patients exhibited increased PD1/TIM-3 expression (not shown), decreased cytokine production (FIG. 41), a 2.5-fold increase in PSGL1 expression (FIG. 42). Taken together, these data demonstrate that CD8+ T-cells in human melanoma patients exhibit characteristics of T-cell exhaustion (e.g., decreased cytokine secretion), increased levels of TCR inhibitory immune checkpoint proteins, and robust elevation of PSGL1, indicating that reducing PSGL1 signaling could be an effective clinical strategy for treating cancer.

FIG. 41 further demonstrates that cytokine production is reduced in CD8+ T-cells from melanoma patients. Representative flow cytometry plots showing IFN-gamma production by CD8+ T-cells from a healthy donor (circles) or melanoma patient (squares) following restimulation with anti-CD3/CD28. Bar graph represents frequency of IFN-gamma+CD8+ T-cells from healthy donors or melanoma patients.

FIG. 42 further demonstrates that CD8+ T-cells from melanoma patients possess higher levels of PSGL1. Histogram of CD162/PSGL1 expression on CD8+ T-cells in 3 representative healthy donors and 3 representative melanoma patients. The relative expression levels of CD 162/PSGL1 in healthy donors (circles) and melanoma patients (squares). Each data point represents an individual donor. Data represents 2 experiments.

### Example 16: Pharmacological PSGL1 Agonist Reduces Cytokine Production and Decreases T_{EFF} Differentiation in CD8+ T-Cells Obtained from Human Donors In Vitro

Previous experiments revealed that PSGL1 ligation promoted CD8+ T-cell exhaustion during LCMV C113 infection and increased YUMM1.5 tumor growth in mice (data not shown). Thus, and herein, experiments were further conducted to determine whether PSGL1 similarly regulates the function of human CD8+ T-cells.

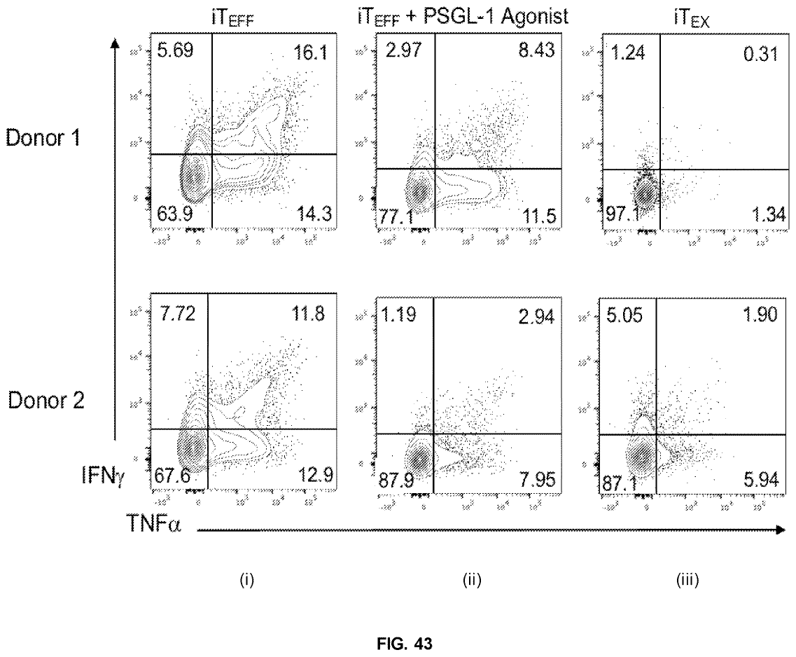

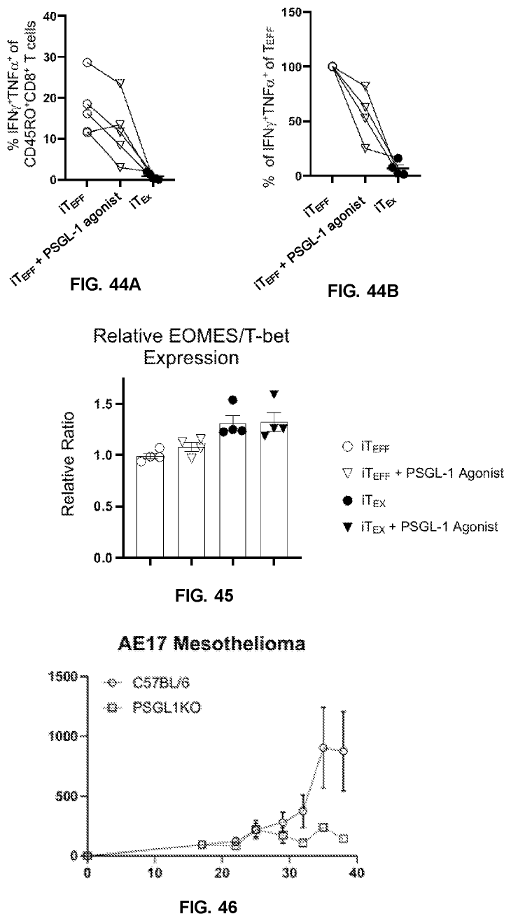

An in vitro model of human T_{EFF} and T_{EX} differentiation was established to measure responses of healthy donor peripheral blood mononuclear cells (PBMCs) to a single (iT_{EFF}) or repeated bouts (iT_{EX}) of TCR stimulation with ImmunoCult Human CD3/CD28 T-cell activator (StemCell). These conditions generate either polyfunctional IFN-gamma- and TNF-alpha-producing CD8+ T-cells (iT_{EFF}) in response to a single TCR stimulation (FIG. 43i) or exhausted CD8+ T-cells (iT_{EX}) that fail to produce cytokines upon restimulation following 6 repeated bouts of TCR stimulation with CD2/CD28 (FIG. 43iii). Restimulation of T-cells after 9 days of culture under iT_{EFF} conditions resulted in CD45RO+CD8+ T-cells with a robust capacity to individually produce and co-produce IFN-gamma and TNF-alpha (FIG. 43iii, FIG. 44A). In contrast, restimulation of T-cells cultured under iT_{EX} conditions failed to produce significant amounts of either cytokine (FIG. 43iii, FIG. 44A).

An in vitro model of human T_{EFF} differentiation was then performed as described with addition of the PSGL1 agonist, full-length 4RA10 (clone KPL-1, 4RA10-FL) to determine if human donor T-cells adopted a T_{EFF} or T_{EX} phenotype under conditions that typically promote T_{EFF} function and active TCR signaling. In 4 out of 5 donor T-cell populations assessed by immunofluorescent flow cytometry, one-time activation of T-cells in the presence anti-PSGL1 (4RA 10-FL) resulted in the decreased ability of T-cells to produce and coproduce IFN-gamma and TNF-alpha despite culture conditions that favor T_{EFF} and enhanced TCR activation. Additionally, the PSGL1 agonist treatment caused reduction of cytokine production that mirror effect of T_{EX} conditions without anti-PSGL1 (4RA10-FL) application (FIG. 43ii, FIG. 44A). For donors in which cytokine production dropped, capacity for coproduction was ~55% of the restimulated effector cells (FIG. 44B).

These data demonstrate that increased PSGL1 signaling alone can override T_{EFF} responses in human CD8+ T-cells, making it a promising therapeutic target for modulating immune responses in patients.

FIG. 43 Representative flow cytometry plots showing IFN-gamma and TNF-alpha production by CD8+ T-cells from two different healthy donors. Plots are pre-gated on live CD8+CD45RO+ cells.

FIG. 44A further depicts a dot plot of the frequencies of IFN-gamma and TNF-alpha double-producing CD8+CD45RO+ cells cultured under iT_{EFF}, iT_{EFF}+PSGL1 agonist, or iT_{EX} conditions. Data are from 3 independent experiments. FIG. 44B further depicts a dot plot showing IFN-gamma and TNF-alpha double-producing CD8+CD45RO+ cells cultured under iT_{EFF} with a PSGL1 agonist antibody or iT_{EX} conditions relative to iT_{EFF} of the same donor for the 4 out of 5 donors which showed reduction of cytokine production upon PSGL1 ligation. For each donor, iT_{EFF} relative frequency was set to 100%. Each data point represents an individual donor. Data are from 3 independent experiments. Lines are connecting results from the same donor.

### Example 17: Pharmacological PSGL1 Agonist Increases EOMES/T-Bet Expression and Decreases T_{EFF} in CD8+ T-Cells Obtained from Human Donors In Vitro

Terminally exhausted T_{EX} express lower levels of T-bet and increased levels of Eomes (compared to T_{EFF}) in chronic LCMV C113 infected mice, in HIV infected patients and in the context of cancer. The in vitro model was employed and the relative Eomes/T-bet expression ratio was measured by immunofluorescent flow cytometry (based on relative MFI), which revealed that the relative Eomes/T-bet expression ratio was increased in iT_{EX} CD8+ T-cells compared to iT_{EFF} T-cells (FIG. 45). Combined with the decreased function of iT_{EX} cells, our results indicate that these in vitro generated T-cells have a phenotype reflective of T_{EX} from patients. This is particularly relevant for human T cells, as TOX expression is not limited to T_{EX}. Consistent with an incomplete differentiation of iT_{EX} from iT_{EFF} when PSGL1 ligation was combined with TCR stimulation, we observed a modest increase in the relative Eomes/T-bet expression ratio (relative MFI) (FIG. 45). When combined with repeated TCR stimulation, the Eomes/T-bet expression ratio of iT_{EX}-treated CD8+ T-cells increased to a similar level as iT_{EFF} without PSGL1 ligation (FIG. 45). These data indicate that signaling via PSGL1 concomitant with TCR stimulation can intrinsically orchestrate the development of T_{EX} from T_{EFF} in human CD8+ T cells.

FIG. 45 further depicts the ratio of EOMES/T-bet expression in CD8+CD45RO+ cultured underiT_{EFF} conditions with PSGL1 agonist antibody, iT_{EX}, or iT_{EX} with PSGL1 agonist conditions relative to iT_{EFF} of the same donor for the 4 out of 5 donors which showed reduction of cytokine production upon PSGL1 ligation. Ratios were calculated using the relative median florescence intensity (MFI) of Eomes+ and/or T-bet+ cells. Each data point represents an individual donor. Data are from 3 independent experiments. Lines are connecting results from the same donor.

### Example 18: Decreased PSGL1 Activation Promotes T_{EFF} in CD8+ T-Cells and Attenuates Mesothelioma Tumor Growth

AE17 mesothelioma tumor volume in WT vs PSGL1−/− mice was measured three times per week over a 38-day period following subcutaneous inoculation with 1×10⁶ AE17 mesothelioma tumor cells. AE17 tumors are also resistant to ICB treatment. PSGL1−/− mice exhibited significantly decreased tumor growth and volume of subcutaneous AE17 mesothelioma tumors (FIG. 46), indicating that reducing PSGL1 activation slows cancerous tumor growth and potentially represents an ICB therapeutic strategy for treatment of mesothelioma, melanoma, and perhaps many other cancers in patients who are non-responsive to current therapies.

FIG. 46 further demonstrates that reduced PSGL1 activity slows cancerous tumor growth. Average tumor growth (volume) of C57BL/6 and PSGL1−/− mice inoculated with AE17 mesothelioma tumor cells over time. Data represent 2 independent experiments.

### Example 19: Generation of a Fab′ Anti-PSGL1 Antagonist Limits the Development of T Cell Exhaustion and Promotes Tumor Growth Control

The available anti-PSGL1 antibody (clone 4RA10, BioXcell) acts as an agonist due to its cross-linking of PSGL1 on T-cells via Fc receptor binding on APCs. Thus, a Fab′ anti-PSGL antibody (anti-PSGL1-Fab′) was created by subjecting the 4RA10-FL to an enzymatic cleavage and further chemical reduction to yield the single-chain anti-PSGL1-Fab′.

### Example 20: Therapeutic PSGL1 Blockade Increase T_{EFF} During Viral Infection In Vivo

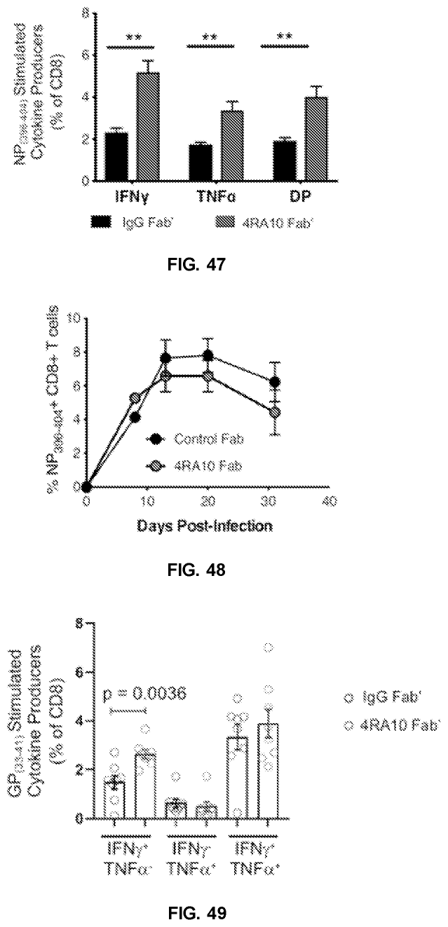

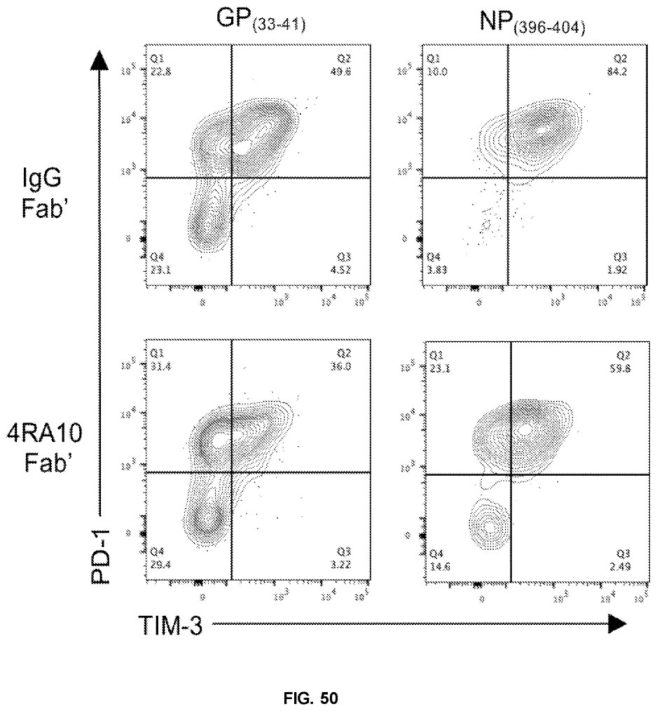

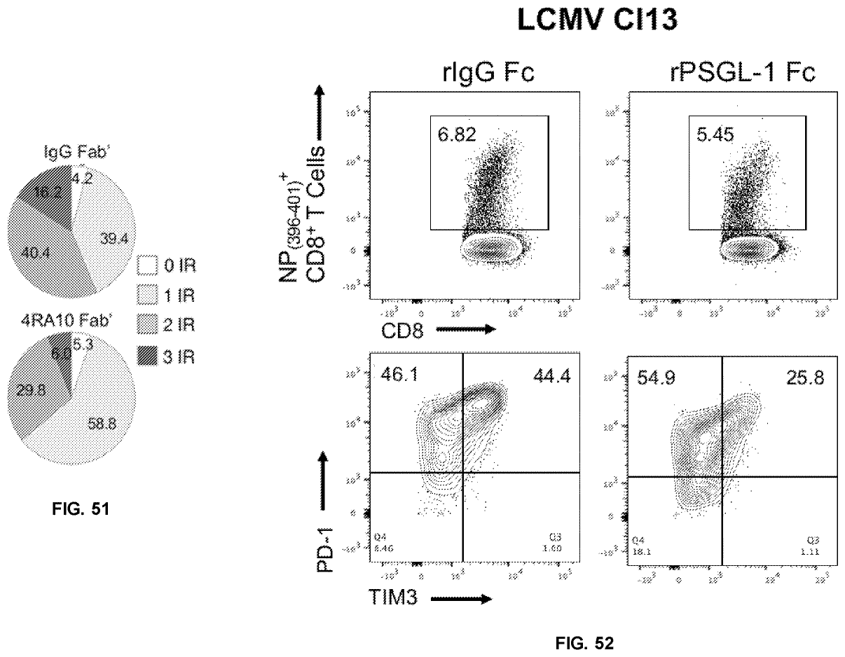

Aforementioned examples demonstrate that viral infection exhausts TCR action and the T_{EFF} response in CD8+ T-cells via enhanced PSGL1 activation. Thus, the anti-PSGL1-Fab′ (4RA10 Fab′) was used to inhibit PSGL1-mediated signaling and boost the T_{EFF} response of CD8+ T-cells. Mice were infected with LCMV CI13 and treated intraperitoneally with Fab′ control rat IgG (control Fab) or anti-PSGL1-Fab′ (4RA10 Fab′) in the morning and afternoon beginning on day 4 after LCMV C113 infection and subsequently on 6, 8, 10, and 12 days post-infection (dpi). Virus-specific NP396-404+ T-cells in the blood were assessed on 8, 13, 20, and 31 dpi. A significantly greater prevalence of IFN-gamma or TNF-alpha single-producer and double-producer cytokine-producing CD8+ T-cells were observed following restimulation with NP 396-401 at 8 dpi (FIG. 47). At 30 dpi, treatment with Fab′ antibodies did not influence the frequency of NP396-404+CD8+ T-cells (FIG. 48). An increase in IFN-gamma-producing CD8+ T-cells was also observed in anti-PSGL1-Fab′-treated mice in response to GP33-41 peptide stimulation at 8 dpi (FIG. 49). Additionally, co-expression of the inhibitory receptors PD1, LAG3, and TIM-3 was decreased in the anti-PSGL1-Fab′-treated group compared to the control group, demonstrating that PSLG-1 blockade reduces expression of T_{EX} biomarkers (FIG. 50, FIG. 51). Taken together, the anti-PSGL1-Fab′ (4RA10 Fab′) effectively promotes immune responses in T-cellsin cellular environments that typically reduce or exhaust T_{EFF}, such as viral infection.

FIG. 47 further demonstrates that Fab′ antibody-mediated PSGL1 blockade increases cytokine production during viral infection. Bar graph of cytokine-producing CD8+ T-cells on 8 dpi from control or 4RA 10 Fab′-anti-PSGL1-treated mice following restimulation with NP (396-404) peptide.

FIG. 48 further depicts that kinetics of the frequency of NP (396-404)-specific CD8+ T-cells in the blood of LCMV C113-infected C57BL/6 mice treated with either control Fab′ or 4RA10 Fab′.

FIG. 49 further depicts mean population quantification of cytokine-producing CD8+ T-cells on 8 dpi from control or 4RA 10 Fab′-treated LCMV C113-infected mice following restimulation with GP (33-41) peptide.

FIG. 50 further depicts representative FACS plots of PD1 and TIM-3 expression in GP (33-41) (left column) and NP (396-401) (right column) virus-specific CD8+ T-cells from LCMV C113-infected mice treated with control Fab′ (top row) or 4RA10 Fab′ (bottom row) at 8 dpi. FIG. 51 further depicts pie charts of combinatorial expression of PD1, LAG3, and TIM-3 on NP (396-404)-specific CD8+ T-cells on 31 dpi in control Fab′- or 4RA 10 Fab′-treated LCMV C113-infected mice. Data were assessed via Boolean gating.

### Example 21: Example 21: Generation of a Recombinant PSGL1 Fusion Protein with a Human IgG1 Fc

Since Fab's have a limited half-life, which could account for the modest effects on T-cell exhaustion, a recombinant PSGL1 with human IgG1 Fc (rPSGL1-Fc) fusion protein was generated. The fusion protein comprises a human Fc region and the ectodomain of mouse PSGL1 that is not able to be fully glycosylated (i.e. lacks a Sialyl-Lewis-X tetrasaccharide) because the rPSGL1-Fc fusion protein was generated in HEK 293 cells which do not express the glycosyltransferases necessary for synthesis of the Sialyl-Lewis-X tetrasaccharide. When expressed in T-cells, the rPSGL1-Fc protein cannot bind to PSGL1 ligands such as P-selectin, E-selectin, L-selectin, or VISTA, which consequently ablates inhibitory PSGL1-mediated signaling and effectively prolongs and reinvigorates the T-cell's TCR activation and its capacity to engage in an effector response.

Recombinant PSGL-1 (rPSGL1-Fc) was generated using the pCR3 plasmid backbone and mouse PSGL-1 and human Fc sequences. rPSGL1-Fc was produced by using transient transfection of plasmid DNA into HEK-293F cells. Expressed protein in supernatants was collected and purified using single-pass chromatography, analyzed for purity and endotoxin levels, and formulated for storage in 1×PBS.

### Example 22: Treatment with rPSGL1-Fc Enhances T_{EFF} and Reduces T_{EX} Biomarkers in T-Cells During Viral Infection

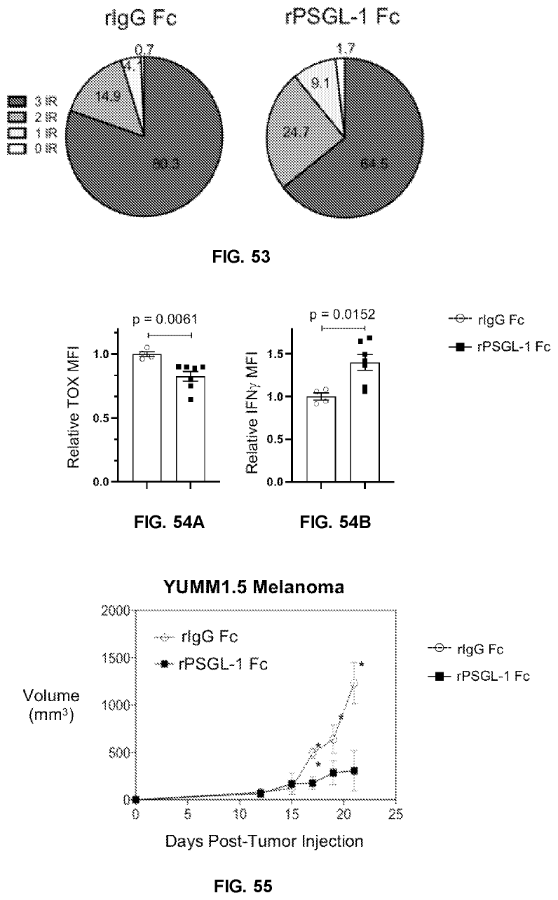

LCMV C113-infected mice were treated with rPSGL1 Fc or control recombinant human IgG1 Fc (rlg Fc) and virus-specific CD8+ T cell responses were assessed at 8 dpi. Although the frequencies of NP396-404+ T cells in the spleens were comparable between rIgG Fc and rPSGL1 Fc treated mice, the frequencies of PD1+ and TIM-3+ double-positive cells were significantly diminished (FIG. 52). Further, overall co-expression of inhibitory receptors was reduced, which is consistent with reduced levels of exhaustion and heightened TCR activation (3 inhibitory receptors: 80.3% vs 64.5%) (FIG. 53). Similar results were observed when WT (CD45.2+) mice receiving WT P14 (CD45.1+) CD8+ T-cells were treated with rIgG Fc or rPSGL1 Fc (data not shown). Additionally, TOX expression was significantly decreased, and IFN-gamma production was increased on a per-cell basis in mice treated with rPSGL1 Fc as measured by MFI with FACs (FIG. 54A, FIG. 54B). Taken together, PSGL1 blockade with rPSGL1-Fc treatment enhances the T-cell effector immune response during T_{EFF}-suppressing conditions.

FIG. 52 further demonstrates that PSGL1-Fc-mediated PSGL1 blockade promotes T_{EFF} and decreases frequencies of PD1+ and TIM-3+ double-producing cells. LCMV C113-infected mice were treated with recombinant human Fc protein (control Fc) or recombinant PSGL1-human Fc protein (rPSGL1-Fc) on days 0, 3 and 6 post-infection (dpi). NP (396-404) virus-specific CD8+ T-cells from the spleens were assessed on 8 dpi (top row). PD1 and TIM-3 expression within NP (396-404)-specific CD8+ T-cells (bottom row). FIG. 53 further demonstrates that rPSGL1-Fc-mediated PSGL1 blockade decreases T-cell exhaustion. Pie charts depicting the co-expression of inhibitory receptors (PD1, LAG3, TIM-3) by NP (396-404) specific CD8+ T-cells. Data were assessed via Boolean gating. FIG. 54A and FIG. 54B further demonstrate that rPSGL1-Fc-mediated PSGL1 blockade decreases TOX expression and increases cytokine production. FIG. 54A further depicts the relative TOX expression (MFI) in TOX+ adoptively transferred WT P14 CD8+ T-cells in LCMV C113-infected mice treated with control Fc (rlgG Fc) or recombinant PSGL1 (rPSGL1 Fc) protein on 0, 3, and 6 dpi. FIG. 54B further depicts the relative IFN-gamma expression (MFI) in IFN-gamma+ adoptively transferred WT P14 CD8+ T-cells as in FIG. 50, following overnight restimulation with GP (33-41) peptide.

### Example 23: Treatment with rPSGL1-Fc Enhances TCR Signaling in T-Cells and Reduces Growth of Malignant Tumors

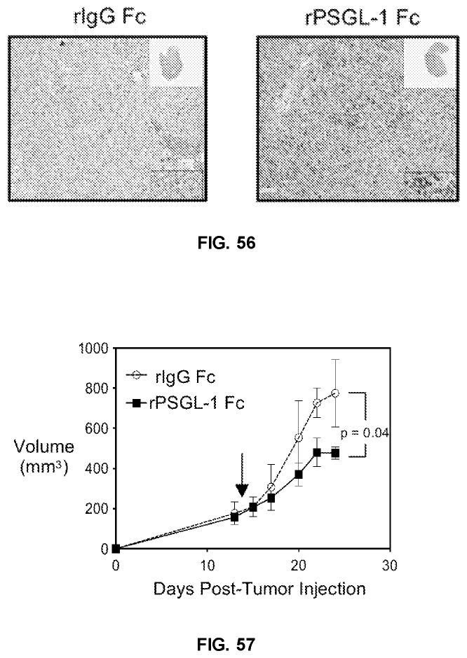

PSGL1 blockade by treatment with rPSGL1-Fc promotes T_{EFF} responses to cancerous tumor microenvironments. WT C57BL/6 mice were inoculated with 5×10⁴ YUMM1.5 melanoma tumor cells via subcutaneous injection. Tumors, spleens, and inguinal draining and non-draining lymph nodes were collected at the indicated time points. Mice were treated 3 times per week with control Fc or rPSGL1 Fc throughout the experiment beginning at day 0 or day 14 post-injection. Tumor volumes were measured blindly 3 times per week. Treatment with rPSGL1-Fc at the time of tumor cell injection resulted in significantly reduced tumor growth that was comparable to that achieved in PSGL1 deficient mice (FIG. 55). Additionally, rPSGL1-Fc treatment increased the infiltration and/or expansion of T-cells within the tumors, as assessed by anti-CD3 staining in YUMM1.5 tumor tissue-sections (FIG. 56). With established tumors after 14 days post-injection of YUMM1.5 cells, PSGL1 blockade reduced tumor growth (FIG. 57). These data demonstrate the feasibility of achieving a therapeutic inhibition PSGL1 signaling in PD1 ICB-resistant cancers.

FIG. 55 further demonstrates that rPSGL1-Fc-mediated PSGL1 blockade reduces cancerous tumor growth rate and shows the average tumor growth (volume) over time in melanoma tumor cell-inoculated mice treated with control Fc (rIgG Fc) or recombinant PSGL1 (rPSGL1 Fc) protein. Data represent 2 independent experiments with 3-5 mice/group per experiment.

FIG. 56 further demonstrates that rPSGL1-Fc-mediated PSGL1 blockade increases TCR activation in cancerous tumors and shows the representative haematoxylin+eosin histology with anti-CD3 staining of YUMM1.5 tumor sections from control Fc and rPSGL1 Fc treated mice collected on day 21. Data represent 2 independent experiments with 3-5 mice/group per experiment.

FIG. 57 further demonstrates that rPSGL1-Fc-mediated PSGL1 blockade reduces growth rate of established cancerous tumors, and the bar graph shows tumor volumes following therapeutic blockade that were measured blindly 3 time per week. Data shown are the averages of tumor growth (volume) with time. Data are representative of 1 independent experiment with 3-7 mice per group.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby

SEQUENCES

| SEQ ID | SEQUENCE | ANNOTATION |
|---|---|---|
| 1 | MPLQLLLLLILLGPGNSLQLWDTWADEA | hPSGL1_FULL |
| | EKALGPLLARDRRQATEYEYLDYDFLPE | |
| | TEPPEMLRNSTDTTPLTGPGTPESTTVE | |
| | PAARRSTGLDAGGAVTELTTELANMGNL | |
| | STDSAAMEIQTTQPAATEAQTTQPVPTE | |
| | AQTTPLAATEAQTTRLTATEAQTTPLAA | |
| | TEAQTTPPAATEAQTTQPTGLEAQTTAP | |
| | AAMEAQTTAPAAMEAQTTPPAAMEAQTT | |
| | QTTAMEAQTTAPEATEAQTTQPTATEAQ | |
| | TTPLAAMEALSTEPSATEALSMEPTTKR | |
| | GLFIPFSVSSVTHKGIPMAASNLSVNYP | |
| | VGAPDHISVKQCLLAILILALVATIFFV | |
| | CTVVLAVRLSRKGHMYPVRNYSPTEMVC | |
| | ISSLLPDGGEGPSATANGGLSKAKSPGL | |
| | TPEPREDREGDDLTLHSFLP | |
| 2 | LQLWDTWADEAEKALGPLLARDRRQATE | hPSGL1_REC |
| | YEYLDYDFLPETEPPEMLRNSTDTTPLT | |
| | GPGTPESTTVEPAARRSTGLDAGGAVTE | |
| | LTTELANMGNLSTDSAAMEIQTTQPAAT | |
| | EAQTTQPVPTEAQTTPLAATEAQTTRLT | |
| | ATEAQTTPLAATEAQTTPPAATEAQTTQ | |
| | PTGLEAQTTAPAAMEAQTTAPAAMEAQT | |
| | TPPAAMEAQTTQTTAMEAQTTAPEATEA | |
| | QTTQPTATEAQTTPLAAMEALSTEPSAT | |
| | EALSMEPTTKRGLFIPFSVSSVTHKGIP | |
| | MAASNLSV | |
| 3 | SLQLQDPWGHETKEAPGPVHLRERRQVV | mPSGL1_REC |
| | GDDDFEDPDYTYNTDPPELLKNVTNTVA | |
| | AHPELPTTVVMLERDSTSAGTSERATEK | |
| | IATTDPTAPGTGGTAVGMLSTDSATQWS | |
| | LTSVETVQPASTEVETSQPAPMEAETSQ | |
| | PAPMEAETSQPAPMEAETSQPAPMEADT | |
| | SQPAPMEADTSKPAPTEAETSKPAPTEA | |
| | ETSQPAPNEAETSKPAPTEAETSKPAPT | |
| | EAETTQLPRIQAVKTLFTTSAATEVPST | |
| | EPTTMETASTESNESTIFLGPSVTHLPD | |
| | LKKGLIVTPGNSPAPTLPGSSD | |
| 4 | VDKTHTCPPCPAPELLGGPSVFLFPPKP | FC |
| | KDTLMISRTPEVTCVVVDVSHEDPEVKF | |
| | NWYVDGVEVHNAKTKPREEQYNSTYRVV | |
| | SVLTVLHQDWLNGKEYKCKVSNKALPAP | |
| | IEKTISKAKGQPREPQVYTLPPSRDELT | |
| | KNQVSLTCLVKGFYPSDIAVEWESNGQP | |
| | ENNYKTTPPVLDSDGSFFLYSKLTVDKS | |
| | RWQQGNVFSCSVMHEALHNHYTQKSLSL | |
| | SPGK | |

1. A fusion polypeptide comprising:
a) a partially glycosylated PSGL1 polypeptide, wherein the partially glycosylated PSGL1 comprises an amino acid sequence having at least 90% sequence identity to SEQ ID NO: 2, and the partially glycosylated PSGL1 polypeptide reduces binding of VISTA to a T-cell; and
b) an Fc polypeptide.

2. The fusion polypeptide of claim 1, wherein the partially glycosylated PSGL1 polypeptide lacks glycosylation at one or more amino acid residues (e.g., serine or threonine).

3. The method of claim 2, wherein the partially glycosylated PSGL1 polypeptide lacks glycosylation at one or more amino acid residues compared to a wild-type PSGL1 polypeptide (e.g., isolated from a T cell expressing the wild-type PSGL1 polypeptide).

4. The fusion polypeptide of claim 1, wherein the partially glycosylated PSGL1 polypeptide lacks one or more glycans.

5. The fusion polypeptide of claim 4, wherein the one or more glycans are O-linked glycans.

6. The fusion polypeptide of claim 4, wherein the one or more glycans comprise a tetrasaccharide.

7. The fusion polypeptide of claim 4, wherein the one or more glycans comprise a Sailyl-Lewis-X tetrasaccharide.

8. The fusion polypeptide of claim 1, wherein the partially glycosylated PSGL1 polypeptide lacks one or more glycans compared to a wild-type PSGL1 polypeptide (e.g., isolated from a T cell expressing the wild-type PSGL1 polypeptide).

9. The fusion polypeptide of claim 1, wherein the partially glycosylated PSGL1 polypeptide comprises an ectodomain PSGL1 polypeptide having a decreased average molecular weight as compared to an average molecular weight of a control ectodomain PSGL1 polypeptide isolated from or expressed by a T-cell (e.g., a T-cell culture).

10. The fusion polypeptide of claim 1, wherein the partially glycosylated PSGL1 polypeptide comprises SEQ ID NO: 2 having a decreased average molecular weight compared to a control PSGL1 polypeptide comprising SEQ ID NO: 2 isolated from or expressed by a T-cell (e.g., a T-cell culture).

11. The fusion polypeptide of claim 1, wherein the partially glycosylated PSGL1 polypeptide comprises an ectodomain PSGL1 polypeptide having a decreased amount (e.g., mean amount) of glycosylation compared to a control ectodomain PSGL1 polypeptide isolated from or expressed by a T-cell (e.g., a T-cell culture).

12. The fusion polypeptide of claim 1, wherein the partially glycosylated PSGL1 polypeptide comprises SEQ ID NO: 2 having a decreased amount (e.g., mean amount) of glycosylation compared to a control PSGL1 polypeptide comprising SEQ ID NO: 2 isolated from or expressed by a T-cell (e.g., a T-cell culture).

13. The fusion polypeptide of claim 1, wherein the glycosylation is measured by mass spectrometry.

14. The fusion polypeptide of claim 1, wherein the glycosylation is measured by molecular weight.

15. The fusion polypeptide of claim 1, wherein the glycosylation is O-linked glycosylation.

16. The fusion polypeptide of claim 1, wherein the glycosylation comprises a tetrasaccharide carbohydrate.

17. The fusion polypeptide of claim 1, wherein the glycosylation comprises a Sailyl-Lewis-X tetrasaccharide.

18. (canceled)

19. (canceled)

20. The fusion polypeptide of claim 1, wherein the partially glycosylated PSGL1 comprises an ectodomain region of PSGL1.

21. (canceled)

22. The fusion polypeptide of claim 1, wherein the partially glycosylated PSGL1 comprises an amino acid sequence as set forth in SEQ ID NO: 2.

23. (canceled)

24. The fusion polypeptide of claim 1, wherein the partially glycosylated PSGL1 polypeptide exhibits reduced (e.g., compared to a wild-type fully glycosylated PSGL1) or no binding to E-selectin and/or L-selectin.

25-48. (canceled)


SEQUENCE LISTING (4 sequences)

SEQ ID NO: 1 - protein, 412 aa, synthetic construct
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 2 - protein, 288 aa, synthetic construct
LQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSV

SEQ ID NO: 3 - protein, 304 aa, synthetic construct
SLQLQDPWGHETKEAPGPVHLRERRQVVGDDDFEDPDYTYNTDPPELLKNVTNTVAAHPELPTTVVMLERDSTSAGTSERATEKIATTDPTAPGTGGTAVGMLSTDSATQWSLTSVETVQPASTEVETSQPAPMEAETSQPAPMEAETSQPAPMEAETSQPAPMEADTSQPAPMEADTSKPAPTEAETSKPAPTEAETSQPAPNEAETSKPAPTEAETSKPAPTEAETTQLPRIQAVKTLFTTSAATEVPSTEPTTMETASTESNESTIFLGPSVTHLPDSGLKKGLIVTPGNSPAPTLPGSSD

SEQ ID NO: 4 - protein, 228 aa, synthetic construct
VDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK